(19) United States
(12) Patent Application Publication
(10) Pub. No.: US 2025/0391548 A1
(43) Pub. Date: Dec. 25, 2025

(54) APPARATUS AND METHODS FOR AUTOMATING PRE-PROCEDURAL COORDINATION WORKFLOWS IN A DIGITAL ENVIRONMENT

(71) Applicant: Qventus, Inc., Mountain View, CA (US)

(72) Inventors: Jeff Mounzer, Sunnyvale, CA (US); Mudit Garg, Mountain View, CA (US); Ian Christopher, Mountain View, CA (US); Chris Diana, Pittsburgh, PA (US); Michael Harris, Kansas City, MO (US); Abhinav Mittal, Palo Alto, CA (US); Karen Miao, Emerald Hills, CA (US); Matthew Garcia, Redondo Beach, CA (US); Garrick Tolley, San Francisco, CA (US); Ben Strahan, Cedar Park, TX (US); Lalit Chawla, Austin, TX (US); Richard Lai, Emeryville, CA (US); Justin Flechsig, Rochester, MN (US)

(73) Assignee: Qventus, Inc., Mountain View, CA (US)

(21) Appl. No.: 18/753,215

(22) Filed: Jun. 25, 2024

(51) Int. Cl. G16H 40/20; G06F 16/2455

(52) U.S. Cl. CPC G16H 40/20; G06F 16/2455

(57) ABSTRACT

Apparatus and methods for automating pre-procedural coordination workflows in a digital environment include at least a processor and a memory communicatively connected to the at least a processor, wherein the memory contains instructions configuring the at least a processor to generate, using a trained content retrieval data structure, a plurality of content retrieval parameters, receive, from a first entity, an input including an input data structure as a function of the plurality of content retrieval parameters, populate, using the input data structure, a content queue comprising a plurality of content elements, query a second entity using at least a content element of the plurality of content elements, update the content queue as a function of at least a query response received from the second entity, generate a recommended course of action as a function of the updated content queue, and perform the recommended course of action.

### FIELD OF THE INVENTION

The present invention generally relates to the field of data management and machine learning. In particular, the present invention is directed to apparatus and methods for automating pre-procedural coordination workflows in a digital environment.

### BACKGROUND

A surgery typically requires a large amount of preparatory work and involves an extensive exchange and maintenance of unstructured data between several entities such as patients, guardians, surgeons, nurses, anesthesiologists, pharmacists, and administrative staff, among others. Such tasks often involve plenty of paperwork and many follow-up phone calls. The extensive and repetitive nature of these tasks may result in errors, delays, outdated information, and miscommunication.

### SUMMARY OF THE DISCLOSURE

In an aspect, an apparatus for automating pre-procedural coordination workflows in a digital environment is described. Apparatus includes a processor and a memory communicatively connected to the processor, wherein the memory contains instructions configuring the processor to generate, using a content retrieval data structure trained on a plurality of training examples, a plurality of content retrieval parameters, wherein generating the plurality of content retrieval parameters includes pretraining the content retrieval data structure on a general set of training examples and retraining the content retrieval data structure on a special set of training examples, wherein the general and the special set of training examples are subsets of the plurality of training examples. Processor is further configured to receive, from a first entity, an input data structure as a function of plurality of content retrieval parameters, populate, using the input data structure, a content queue comprising a plurality of content elements, query at least a second entity using at least a content element of the plurality of content elements, update the content queue as a function of at least a query response received from the at least a second entity, generate a recommended course of action as a function of the updated content queue, and perform the recommended course of action.

In another aspect, a method for automating pre-procedural coordination workflows in a digital environment is described. Method is performed by processor and includes generating, using content retrieval data structure trained on plurality of training examples, plurality of content retrieval parameters, wherein generating the plurality of content retrieval parameters includes pretraining the content retrieval data structure on general set of training examples and retraining the content retrieval data structure on special set of training examples, wherein the general and the special set of training examples are subsets of the plurality of training examples. Method further includes receiving, from first entity, input data structure as a function of plurality of content retrieval parameters, populating, using the input data structure, content queue comprising plurality of content elements, querying at least a second entity using at least a content element of the plurality of content elements, updating the content queue as a function of at least a query response received from the second entity, generating recommended course of action as a function of the updated content queue, and performing the recommended course of action.

These and other aspects and features of nonlimiting embodiments of the present invention will become apparent to those skilled in the art upon review of the following description of specific nonlimiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is an illustration of an exemplary embodiment of an apparatus for generating structured pre-procedural coordination workflow data;

FIG. 2 is a box diagram of an exemplary embodiment of an input data structure;

FIG. 3 is a block diagram of an exemplary embodiment of a machine learning process;

FIG. 4 is a block diagram of an exemplary embodiment of a neural network;

FIG. 5 is a block diagram of an exemplary embodiment of a node of a neural network;

FIG. 6 is an illustration of an exemplary embodiment of fuzzy set comparison;

FIG. 7 is a flow diagram of an exemplary embodiment of a method for maintaining retrieved contents;

FIG. 8 is an exemplary embodiment of a chatbot system;

FIGS. 9A-Z are exemplary embodiments of graphical user interfaces; and

FIG. 10 is a block diagram of an exemplary embodiment of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to apparatus and methods for automating pre-procedural coordination and pre-admission testing workflows in a digital environment. In one or more embodiments, apparatus may include a processor and a memory communicatively connected to the processor, wherein the memory contains instructions configuring the processor to generate, using at least a content retrieval data structure such as at least a content retrieval machine learning model trained on a plurality of training examples, a plurality of content retrieval parameters. Processor is further configured to receive, from a first entity, such as a patient scheduled for a medical procedure, an input data structure as a function of plurality of content retrieval parameters, and populate, using the input data structure, a content queue comprising a plurality of content elements. Processor is further configured to query at least a second entity, such as surgeons, nurses, anesthesiologists, pharmacists, nutritionists, and/or administrative staff affiliated with one or more medical facilities, using at least a content element of plurality of content elements. Processor is further configured to receive, from at least a second entity, at least a query response, such as an order of prescription or a confirmation of appointment, as a function of the query, and update content queue as a function of the at least a query response. Processor is further configured to generate a recommended course of action, such as medications to start or stop using before a medical procedure, among other precautions, as a function of updated content queue, and perform the recommended course of action.

Aspects of the present disclosure may be used to streamline and structure bidirectional transmission of data and reduce the workload of involved entities. Aspects of the present disclosure may be used to reduce human errors in data maintenance. Aspects of the present disclosure may be used to identify alternative operations with superior expected outcomes. Exemplary embodiments illustrating aspects of the present disclosure are described below in the context of several specific examples.

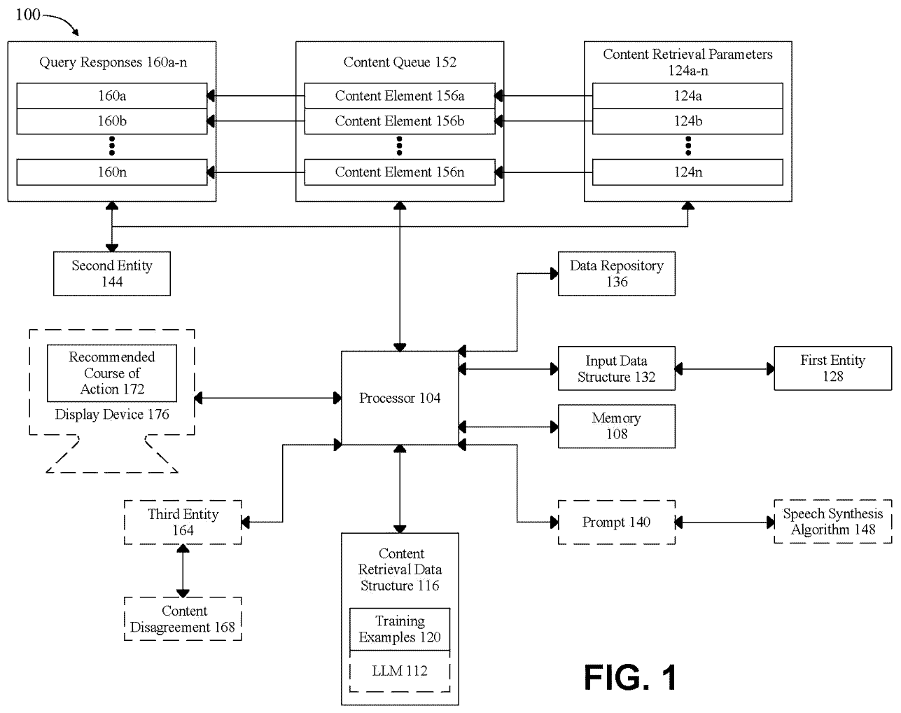

Referring now to FIG. 1, an apparatus 100 for automating pre-procedural coordination workflows in a digital environment is illustrated. For the purposes of this disclosure, “pre-procedural coordination workflow” is an action or a series of actions performed to coordinate a medical procedure. Pre-procedural coordination workflow may include a pre-admission testing workflow. For the purposes of this disclosure, a “pre-admission testing workflow” is an action or a series of actions performed to collect pre-admission testing data. For the purposes of this disclosure, “pre-admission testing data” are data recorded prior to an individual's hospitalization and used for performing one or more medical procedures on the individual. For the purposes of this disclosure, a “digital environment” is an integrated communications environment where digital devices communicate and manage data and interactions within the digital environment. Digital device may include any computing device as described in this disclosure. Additionally, any processing step described in this disclosure may be performed in digital environment. For example, digital environment may be one of a computer system, computer network, and the like. In an exemplary embodiment, the digital environment may include a plurality of remote devices, as described in detail below in this disclosure. In some embodiments, digital environment may also include any electronically based asset associated with the digital environment. For example, electronically based digital assets may be computer programs, data, data stores, and the like, but are not limited to such examples. Digital environment may be connected to a processor by a network. Digital environment may employ any type of network architecture. For example, digital environment may employ a peer-to-peer (P2P) architecture where each computing device in a computing network is connected with every computing device in the network and every computing device acts as a server for the data stored in the computing device. In a further exemplary embodiment, digital environment may also employ a client server architecture where a computing device is implemented as a central computing device (e.g., server) that is connected to each client computing device and communication is routed through the central computing device. However, network architecture is not limited thereto. Further, any network topology may be used. For example, digital environment may employ a mesh topology where a computing device is connected to one or multiple other computing devices using point-to-point connections. However, network topology is not limited thereto. A person of ordinary skill in the art will be able to recognize the various network architectures that may be employed by digital environment upon reviewing the entirety of this disclosure.

With continued reference to FIG. 1, apparatus 100 includes a processor 104. In one or more embodiments, processor 104 may include a computing device. Computing device could include any analog or digital control circuit, including an operational amplifier circuit, a combinational logic circuit, a sequential logic circuit, an application-specific integrated circuit (ASIC), a field programmable gate arrays (FPGA), or the like. Computing device may include a processor communicatively connected to a memory, as described below. Computing device may include any computing device as described in this disclosure, including without limitation a microcontroller, microprocessor, digital signal processor, and/or system on a chip as described in this disclosure. Computing device may include, be included in, and/or communicate with a mobile device such as a mobile telephone, smartphone, or tablet. Computing device may include a single computing device operating independently, or may include two or more computing device operating in concert, in parallel, sequentially, or the like; two or more computing devices may be included together in a single computing device or in two or more computing devices. Computing device may interface or communicate with one or more additional devices as described below in further detail via a network interface device. Network interface device may be utilized for connecting computing device to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus, or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device may include but is not limited to, for example, a first computing device or cluster of computing devices in a first location and a second computing device or cluster of computing devices in a second location. Computing device may include one or more computing devices dedicated to data storage, security, distribution of traffic for load balancing, and the like. Computing device may distribute one or more computing tasks as described below across a plurality of computing devices of computing device, which may operate in parallel, in series, redundantly, or in any other manner used for distribution of tasks or memory between computing devices. Computing device may be implemented, as a nonlimiting example, using a “shared nothing” architecture.

With continued reference to FIG. 1, computing device may be designed and/or configured to perform any method, method step, or sequence of method steps in any embodiment described in this disclosure, in any order and with any degree of repetition. For instance, computing device may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. A person of ordinary skill in the art, upon reviewing the entirety of this disclosure, will be aware of various ways in which steps, sequences of steps, processing tasks, and/or data may be subdivided, shared, or otherwise dealt with using iteration, recursion, and/or parallel processing. More details regarding computing devices will be described below.

With continued reference to FIG. 1, apparatus 100 includes a memory 108 communicatively connected to processor 104, wherein the memory 108 contains instructions configuring the processor 104 to perform any processing steps described herein. For the purposes of this disclosure, “communicatively connected” means connected by way of a connection, attachment, or linkage between two or more relata which allows for reception and/or transmittance of information therebetween. For example, and without limitation, this connection may be wired or wireless, direct, or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. A communicative connection may be achieved, for example and without limitation, through wired or wireless electronic, digital, or analog, communication, either directly or by way of one or more intervening devices or components. Further, communicative connection may include electrically coupling or connecting at least an output of one device, component, or circuit to at least an input of another device, component, or circuit. For example, and without limitation, using a bus or other facility for intercommunication between elements of a computing device. Communicative connecting may also include indirect connections via, for example and without limitation, wireless connection, radio communication, low-power wide-area network, optical communication, magnetic, capacitive, or optical coupling, and the like. In some instances, the terminology “communicatively coupled” may be used in place of communicatively connected in this disclosure.

With continued reference to FIG. 1, computing device may perform determinations, classification, and/or analysis steps, methods, processes, or the like as described in this disclosure using machine learning processes. For the purposes of this disclosure, a “machine learning process” is a process that automatedly uses a body of data known as “training data” and/or a “training set” to generate an algorithm that will be performed by a processor module to produce outputs given data provided as inputs; this is in contrast to a nonmachine learning software program where the commands to be executed are determined in advance by a user and written in a programming language. A machine learning process may utilize supervised, unsupervised, lazy-learning processes and/or neural networks. As a nonlimiting example, apparatus 100 may implement a large language model (LLM) 112 to extract and process textual data. More details regarding computing devices and machine learning processes will be provided below.

With continued reference to FIG. 1, apparatus 100 may include or be communicatively connected to a database. For the purposes of this disclosure, a “database” is an organized collection of data or a type of data store based on the use of a database management system (DBMS), the software that interacts with end users, applications, and the database itself to capture and analyze the data. Database may be implemented, without limitation, as a relational database, a key-value retrieval database such as a NoSQL database, or any other format or structure for use as database that a person of ordinary skill in the art would recognize as suitable upon review of the entirety of this disclosure. Database may alternatively or additionally be implemented using a distributed data storage protocol and/or data structure, such as a distributed hash table or the like. Database may include a plurality of data entries and/or records as described in this disclosure. Data entries in database may be flagged with or linked to one or more additional elements of information, which may be reflected in data entry cells and/or in linked tables such as tables related by one or more indices in database or another relational database. A person of ordinary skill in the art, upon reviewing the entirety of this disclosure, will be aware of various ways in which data entries in database may store, retrieve, organize, and/or reflect data and/or records as used herein, as well as categories and/or populations of data consistently with this disclosure.

With continued reference to FIG. 1, apparatus 100 may include or be communicatively connected to one or more electronic health records (EHRs). For the purposes of this disclosure, an electronic health record (EHR) is a comprehensive collection of records relating to the health history, diagnosis, or condition of patient, relating to treatment provided or proposed to be provided to the patient, or relating to additional factors that may impact the health of the patient; elements within an EHR, once combined, may provide a detailed picture of patient's overall health. In one or more embodiments, one or more user inputs and/or one or more medical features determined therefrom may be deposited to and retrieved from one or more EHRs. In one or more embodiments, EHR may include demographic data of patient; for example, and without limitation, EHR may include basic information about patient such as name, age, gender, ethnicity, socioeconomic status, and/or the like. In one or more embodiments, each EHR may also include patient's medical history; for example, and without limitation, EHR may include a detailed record of patient's past health conditions, medical procedures, hospitalizations, and illnesses such as surgeries, treatments, medications, allergies, and/or the like. In one or more embodiments, each EHR may include lifestyle information of patient; for example, and without limitation, EHR may include details about the patient's diet, exercise habits, smoking and alcohol consumption, and other behaviors that could impact patient's health. In one or more embodiments, EHR may include patient's family history; for example, and without limitation, EHR may include a record of hereditary diseases. In one or more embodiments, database may comprise a plurality of EHRs. In one or more embodiments, EHRs may be retrieved from a repository of similar nature as database.

With continued reference to FIG. 1, processor 104 is configured to generate, using at least a content retrieval data structure 116 trained on a plurality of training examples 120, a plurality of content retrieval parameters 124a-n. For the purposes of this disclosure, a “content retrieval data structure” is a data structure configured to generate a plurality of content retrieval parameters 124a-n that may be used to isolate medically relevant information from a dataset. For the purposes of this disclosure, a “content retrieval parameter” is a parameter that may be used to selectively isolate one or more data elements from a set of data elements. Content retrieval parameter 124a-n may include any parameter that may help identify or communicate with an individual and/or any parameter that may impact the health of an individual, consistent with details described above for EHRs, such as without limitation name, address, contact information, insurance information, age, height, weight, race and ethnicity, gender, dietary habits, body fat, cholesterol level, allergies, blood pressure, use of alcohol, tobacco, drugs, or medications, as well as any prior diagnostic results and hospitalization history. For the purposes of this disclosure, a “data structure” is a format of data organization, management, and storage that is usually chosen for efficient access to data. Content retrieval data structure may include any type of data structure recognized by a person of ordinary skill in the art upon reviewing the entirety of this disclosure, such as without limitation, stack, queue, array, list, or tree. In some cases, at least a content retrieval data structure 116 may include a machine learning model such as a content retrieval machine learning model, a generative model, and/or otherwise implement one or more types of artificial intelligence (AI) algorithms, as described below in this disclosure. As nonlimiting examples, at least a content retrieval data structure 116 may include a large language model, as described in this disclosure, configured to perform functions including extraction of textual data from digital files, synthesizing prompts in one or more complete sentences or paragraphs using one or more keywords or input elements, and transcribing audio data into textual data (i.e., speech recognition), among others. Alternatively, and/or additionally, at least a content retrieval data structure 116 may include models or algorithms, such as without limitation OCR and computer vision, that analyze, transform, organize, and/or summarize graphical data. Additional details will be described in this disclosure.

With continued reference to FIG. 1, training examples 120 may include any type of data including texts, images, audios, videos, one or more combinations thereof, and/or the like, and may be retrieved from any source deemed suitable by a person of ordinary skill in the art upon reviewing the entirety of this disclosure. As nonlimiting examples, training examples 120 may include textual files such as clinical notes prepared by medical professionals, diagnostic results provided by medical facilities, and publicly available findings in data repositories, newspapers, medical journals, conference proceedings, trade show flyers, textbooks, and/or the like. Additionally, and/or alternatively, training examples 120 may contain images including medical images such as digital photographs, optical images including X-ray images, computed tomography (CT) scans, magnetic resonance imaging (MRI) data, ultrasound images, electrocardiograms (ECG), and/or the like. Additionally, and/or alternatively, training examples 120 may include audio data collected across and/or synthesized based on a diverse population, capturing various languages, dialects, accents, and grammatical preferences.

With continued reference to FIG. 1, generating plurality of content retrieval parameters 124a-n comprises pretraining the at least a content retrieval data structure 116 on a general set of training examples 120 and retraining the at least a content retrieval data structure 116 on a special set of training examples 120, wherein the general and the special set of training examples 120 are subsets of the plurality of training examples. As a nonlimiting example, content retrieval data structure may first be trained using a general collection of medical literature, such as a collection scientific journal articles published in Cell in 2023, and subsequently retrained/fine-tuned using a subset thereof pertaining to a specific discipline such as urology.

With continued reference to FIG. 1, in one or more embodiments, processor 104 may be configured to generate a plurality of content retrieval data structures 116, each of which is configured to perform a distinct task in a pre-procedural coordination or pre-admission testing context. In some cases, each content retrieval data structure 116 of plurality of content retrieval data structures may include a distinct machine learning model. In some cases, each content retrieval data structure 116 of plurality of content retrieval data structures may function as a separate “agent”. As a nonlimiting example, a first content retrieval data structure 116 may be configured to process demographic information such as age and ethnicity from EHRs, a second content retrieval data structure 116 may be configured to isolate date-related information by processing optical characters within digital files, and a third content retrieval data structure may be configured to use natural language processing tools to detect medical conditions or symptoms from a set of audio data. In such cases, each content retrieval data structure 116 of plurality of content retrieval data structures 116 may be specifically trained for the particular task it is targeted towards, following the procedures described in this disclosure. As another nonlimiting example, a first content retrieval data structure 116 may be configured to assist a patient, a second content retrieval data structure 116 may be configured to assist a surgeon, and a third content retrieval data structure may be configured to represent a pre-admission testing nurse that coordinates between the patient and the surgeon, consistent with details described below.

With continued reference to FIG. 1, in one or more embodiments, one or more machine learning models may be used to perform certain function or functions of apparatus 100, such as generating content retrieval parameters 124a-n, as described above. Processor 104 may use a machine learning module to implement one or more algorithms as described herein or generate one or more machine learning models, such as at least a content retrieval data structure 116, as described above. However, machine learning module is exemplary and may not be necessary to generate one or more machine learning models and perform any machine learning described herein. In one or more embodiments, one or more machine learning models may be generated using training data. Training data may include inputs and corresponding predetermined outputs so that machine learning model may use correlations between the provided exemplary inputs and outputs to develop an algorithm and/or relationship that then allows the machine learning model to determine its own outputs for inputs. Training data may contain correlations that a machine learning process may use to model relationships between two or more categories of data elements. Exemplary inputs and outputs may be retrieved from a database, extracted from medical literature, selected from one or more EHRs, synthesized using one or more generative models, or be provided by a user. In one or more embodiments, machine learning module may obtain training data by querying a communicatively connected database that includes past inputs and outputs. Training data may include inputs from various types of databases, resources, and/or user inputs and outputs correlated to each of those inputs, so that machine learning model may determine an output. Correlations may indicate causative and/or predictive links between data, which may be modeled as relationships, such as mathematical relationships, by machine learning models, as described in further detail below. In one or more embodiments, training data may be formatted and/or organized by categories of data elements by, for example, associating data elements with one or more descriptors corresponding to categories of data elements. As a nonlimiting example, training data may include data entered in standardized forms by persons or processes, such that entry of a given data element in a given field in a form may be mapped to one or more descriptors of categories. Elements in training data may be linked to descriptors of categories by tags, tokens, or other data elements. In one or more embodiments, training data may include previous outputs such that one or more machine learning models may iteratively produce outputs.

With continued reference to FIG. 1, in one or more embodiments, processor 104 may implement one or more aspects of “generative artificial intelligence (AI)”, a type of AI that uses machine learning algorithms to create, establish, or otherwise generate data such as, without limitation, interpretations of medical data. In one or more embodiments, machine learning module described in this disclosure may generate one or more generative machine learning models that are trained on one or more prior iterations. One or more generative machine learning models may be configured to generate new examples that are similar to the training data of the one or more generative machine learning models but are not exact replicas; for instance, and without limitation, data quality or attributes of the generated examples may bear a resemblance to the training data provided to one or more generative machine learning models, wherein the resemblance may pertain to underlying patterns, features, or structures found within the provided training data.

With continued reference to FIG. 1, in some cases, generative machine learning models may include one or more generative models. For the purposes of this disclosure, a “generative model” is a statistical model of joint probability distribution P(X, Y) on a given observable variable, x, representing features or data that can be directly measured or observed (e.g., training examples) and target variable, y, representing outcomes or labels that one or more generative models aim to predict or generate (e.g., plurality of content retrieval parameters 124a-n, among others). Exemplary generative models include generative adversarial models (GANs), diffusion models, and the like. In one or more embodiments, generative models may rely on Bayes theorem to find joint probability; for instance, and without limitation, naive Bayes classifiers may be employed by computing device to categorize input data such as, without limitation, texts and images from training examples into difference categories.

With continued reference to FIG. 1, in a nonlimiting example, one or more generative machine learning models may include one or more naive Bayes classifiers generated by processor 104, using a naive Bayes classification algorithm. Naïve Bayes classification algorithm may generate classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naive Bayes classification algorithm may be based on Bayes Theorem expressed as P(A/B)=P(B/A)×P(A)+P(B), where P(A/B) is the probability of hypothesis A given data B, also known as posterior probability; P(B/A) is the probability of data B given that the hypothesis A was true; P(A) is the probability of hypothesis A being true regardless of data, also known as prior probability of A; and P(B) is the probability of data regardless of the hypothesis. A naive Bayes algorithm may be generated by first transforming training data into a frequency table. Processor 104 and/or computing device may then calculate a likelihood table by calculating probabilities of different data entries and classification labels. Processor 104 and/or computing device may utilize a naive Bayes equation to calculate a posterior probability for each class. A class containing the highest posterior probability is the outcome of prediction.

With continued reference to FIG. 1, although naive Bayes classifier may be primarily known as a probabilistic classification algorithm; however, it may also be considered a generative model described herein due to its capability of modeling the joint probability distribution P(X, Y) over observable variables, X, and target variable, Y. In one or more embodiments, naive Bayes classifier may be configured to make an assumption that the features, X, are conditionally independent given class label, Y, allowing generative model to estimate a joint distribution as P(X, Y)=P(Y)ΠiP(Xᵢ|Y), wherein P(Y) is the prior probability of the class, and P(Xᵢ|Y) is the conditional probability of each feature given the class. One or more generative machine learning models containing naive Bayes classifiers may be trained on labeled training data, estimating conditional probabilities P(Xᵢ|Y) and prior probabilities P(Y) for each class; for instance, and without limitation, using techniques such as Maximum Likelihood Estimation (MLE). One or more generative machine learning models containing naive Bayes classifiers may select a class label, y, according to prior distribution, P(Y), and for each feature, Xᵢ, sample at least a value according to conditional distribution, P(Xᵢ|y). Sampled feature values may then be combined to form one or more new data instances with selected class label, y. In a nonlimiting example, one or more generative machine learning models may include one or more naive Bayes classifiers to generate new examples of content retrieval parameters 124a-n, as a function of exemplary input data or classes of input data such as, without limitation, training examples and elements such as text and images therein, or the like, wherein the models may be pretrained and/or retrained using a plurality of features within training examples, as described herein as input correlated to plurality of labelled classes, such as content retrieval parameters 124a-n, as outputs.

With continued reference to FIG. 1, in one or more embodiments, one or more generative machine learning models may include generative adversarial network (GAN). For the purposes of this disclosure, a “generative adversarial network” is a type of artificial neural network with at least two sub models (i.e., neural networks), a generator and a discriminator, that compete against each other in a process that ultimately results in the generator learning to generate new data samples, wherein the “generator” is a component of the GAN that learns to create hypothetical data by incorporating feedback from the “discriminator” configured to distinguish real data from the hypothetical data. In one or more embodiments, generator may learn to make discriminator classify its output as real. In one or more embodiments, discriminator may include a supervised machine learning model while generator may include an unsupervised machine learning model, as described in further detail below.

With continued reference to FIG. 1, in one or more embodiments, discriminator may include one or more discriminative models, i.e., models of conditional probability P(Y|X=x) of target variable, Y, given observed variable, X. In one or more embodiments, discriminative models may learn boundaries between classes or labels in given training data. In a nonlimiting example, discriminator may include one or more classifiers as described in further detail below to distinguish between different categories, e.g., real vs. fake, or states, e.g., TRUE vs. FALSE within the context of generated data such as, without limitations, content retrieval parameters 124a-n. In one or more embodiments, processor 104 may implement one or more classification algorithms such as, without limitation, Support Vector Machines (SVM), Logistic Regression, Decision Trees, and/or the like to define decision boundaries.

With continued reference to FIG. 1, in a nonlimiting example, generator of GAN may be responsible for creating synthetic data, such as synthetic training examples or training images, that resemble true training examples or training images collected from a clinical setting by one or more medical professionals. In one or more embodiments, GAN may be configured to training examples and generate corresponding synthetic training examples containing information describing or evaluating one or more content retrieval parameters 124a-n. On the other hand, discriminator of GAN may evaluate the authenticity of the generated content by comparing it to real training examples; for example, discriminator may distinguish between genuine and generated content and provide feedback to generator to improve the model performance. Additionally, and/or alternatively, GAN may include a conditional GAN as an extension of the basic GAN as described herein that allows for generation of synthetic training examples based on certain labels. In standard GAN, generator may produce samples from random noise, whereas in conditional GAN, generator may produce samples based on random noise and a given condition or label.

With continued reference to FIG. 1, additionally or alternatively, one or more generative models may also include a variational autoencoder (VAE). For the purposes of this disclosure, a “variational autoencoder” is an autoencoder (i.e., an artificial neural network architecture) whose encoding distribution is regularized during the model training process in order to ensure that its latent space includes desired properties allowing new data sample generation. In one or more embodiments, VAE may include a prior and noise distribution respectively, trained using expectation-maximization meta-algorithms such as, without limitation, probabilistic PCA, sparse coding, among others. In a nonlimiting example, VAE may use a neural network as an amortized approach to jointly optimize across input data and output a plurality of parameters for corresponding variational distribution as it maps from a known input space to a low-dimensional latent space. Additionally, or alternatively, VAE may include a second neural network, for example, and without limitation, a decoder, wherein the “decoder” is configured to map from latent space to input space.

With continued reference to FIG. 1, in a nonlimiting example, VAE may be used by processor 104 and/or computing device to model complex relationships between various parts of training data. In some cases, VAE may encode input data into a latent space, capturing one or more content retrieval parameters 124a-n. Such encoding process may include learning one or more probabilistic mappings from observed training data to a lower-dimensional latent representation. Latent representation may then be decoded back into the original data space, therefore reconstructing the training data. In some cases, such decoding process may allow VAE to generate new examples or variations that are consistent with the learned distributions.

With continued reference to FIG. 1, in one or more embodiments, Additionally, or alternatively, one or more generative machine learning models may utilize one or more predefined templates representing, for example, and without limitation, acceptable content retrieval parameters 124a-n. In a nonlimiting example, one or more template content retrieval parameters 124a-n (i.e., predefined parameters associated with common medical characteristics such as such as a blood pressure between 50-200 mmHg, a blood sugar level between 80-200 mg/dL, a triglyceride level between 100 and 1000 mg/dL, among others) may serve as benchmarks for comparing and evaluating content retrieval parameters 124a-n.

With continued reference to FIG. 1, processor 104 and/or computing device may configure generative machine learning models to analyze input data such as, without limitation, training images to one or more predefined templates such as exemplary training images representing one or more common medical traits, thereby allowing processor 104 and/or computing device to identify discrepancies or deviations from exemplary training images. Such commonly observed medical traits may include, for example, structural or anatomic traits such as size, shape, the number of blood vessels, or the like, of one or more organs such as hearts, lungs, livers, among others. In some cases, processor 104 and/or computing device may be configured to pinpoint specific errors in training images. In a nonlimiting example, processor 104 and/or computing device may be configured to implement generative machine learning models to incorporate additional models to detect additional content retrieval parameters 124a-n. In some cases, errors may be classified into different categories or severity levels. In a nonlimiting example, some errors may be considered minor, and generative machine learning model such as, without limitation, GAN may be configured to generate revisions suggesting only slight adjustments while others may be more significant and demand more substantial corrections. In some embodiments, processor 104 and/or computing device may be configured to flag or highlight one or more flaws in training examples, altering one or more elements therein using one or more generative machine learning models described in this disclosure. In some cases, one or more generative machine learning models may be configured to generate and output indicators such as, without limitation, visual indicator, and/or the like. Such indicators may be used to signal the detected error described herein.

With continued reference to FIG. 1, in one or more embodiments, processor 104 and/or computing device may be configured to identify, and rank detected common flaws (e.g., structural flaws in anatomy) across a plurality of training examples; for instance, and without limitation, one or more machine learning models may classify flaws in a specific order, such as a descending order from the most to the least relevant, from the most to the least severe, or from the most to the least common. Such ranking process may enable a prioritization of most prevalent issues, allowing users to address these flaws.

With continued reference to FIG. 1, in one or more embodiments, one or more generative machine learning models may also be applied by processor 104 and/or computing device to edit, modify, or otherwise manipulate existing data or data structures. In one or more embodiments, output of training data used to train one or more generative machine learning models, such as GAN as described herein, may include exemplary training images that visually demonstrate one or more revisions made, such as an altered structure, geometry, dimension, or the like. In some cases, revised training examples or images may be overlayed on top of an original training example or image or displayed side by side with the original training example or image.

With continued reference to FIG. 1, in one or more embodiments, processor 104 and/or computing device may be configured to continuously monitor user inputs submitted by users. In one or more embodiments, processor 104 may configure discriminator to provide ongoing feedback and further corrections as needed to subsequent input data. An iterative feedback loop may be created as processor 104 continuously receives real-time data, identifies errors (e.g., discrepancies between received training examples and one or more standard or acceptable training examples) as a function of real-time data, delivering corrections based on the identified errors and monitoring subsequent model outputs and/or user feedback on the delivered corrections. In one or more embodiments, processor 104 may be configured to retrain one or more generative machine learning models based on user modified/annotated training examples or update training data of one or more generative machine learning models by integrating revised training examples into original training data. In such embodiment, iterative feedback loop may allow image generator to adapt to user's needs and performance requirements, enabling one or more generative machine learning models described herein to learn and update based on user responses and generated feedback.

With continued reference to FIG. 1, other exemplary embodiments of generative machine learning models may include, without limitation, long short-term memory networks (LSTMs), (generative pre-trained) transformer (GPT) models, mixture density networks (MDN), and/or the like. A person of ordinary skill in the art, upon reviewing the entirety of this disclosure, will be aware of various generative machine learning models that may be used to perform certain function or functions of apparatus 100, such as generating content retrieval parameters 124a-n, as described herein.

With continued reference to FIG. 1, in one or more embodiments, machine learning module may be further configured to generate a multimodal neural network that combines various neural network architectures described herein. In a nonlimiting example, multimodal neural network may combine LSTM for time-series analysis with GPT models for natural language processing. Such fusion may be applied by processor 104 and/or computing device to generate synthetic training examples, content retrieval parameters 124a-n, and/or the like. In one or more embodiments, multimodal neural network may also include a hierarchical multimodal neural network, wherein the hierarchical multimodal neural network may involve a plurality of layers of integration. For instance, and without limitation, different models may be combined at various stages of the network. Convolutional neural network (CNN) may be used for image feature extraction, followed by LSTMs for sequential pattern recognition, and a MDN at the end for probabilistic modeling. Other exemplary embodiments of multimodal neural network may include, without limitation, ensemble-based multimodal neural network, cross-modal fusion, adaptive multimodal network, among others. A person of ordinary skill in the art, upon reviewing the entirety of this disclosure, will be aware of various multimodal neural networks and combination thereof that may be implemented by apparatus 100 in accordance with this disclosure.

With continued reference to FIG. 1, in one or more embodiments, processor 104 may perform one or more functions of apparatus 100, such as extraction of one or more features from training examples or inputs, by using optical character recognition (OCR) to read digital files and extract information therein. In one or more embodiments, OCR may include automatic conversion of images (e.g., typed, handwritten, or printed text) into machine-encoded text. In one or more embodiments, recognition of at least a keyword from an image component may include one or more processes, including without limitation OCR, optical word recognition, intelligent character recognition, intelligent word recognition, and the like. In one or more embodiments, OCR may recognize written text one glyph or character at a time, for example, for languages that use a space as a word divider. In one or more embodiments, intelligent character recognition (ICR) may recognize written text one glyph or character at a time, for instance by employing machine learning processes. In one or more embodiments, intelligent word recognition (IWR) may recognize written text, one word at a time, for instance by employing machine learning processes.

With continued reference to FIG. 1, in one or more embodiments, OCR may employ preprocessing of image components. Preprocessing process may include without limitation de-skew, de-speckle, binarization, line removal, layout analysis or “zoning”, line and word detection, script recognition, character isolation or “segmentation”, and normalization. In one or more embodiments, a de-skew process may include applying a transform (e.g., homography or affine transform) to an image component to align text. In one or more embodiments, a de-speckle process may include removing positive and negative spots and/or smoothing edges. In one or more embodiments, a binarization process may include converting an image from color or greyscale to black-and-white (i.e., a binary image). Binarization may be performed as a simple way of separating text (or any other desired image component) from the background of image component. In one or more embodiments, binarization may be required for example if an employed OCR algorithm only works on binary images. In one or more embodiments, line removal process may include removal of non-glyph or non-character imagery (e.g., boxes and lines). In one or more embodiments, a layout analysis or “zoning” process may identify columns, paragraphs, captions, and the like as distinct blocks. In one or more embodiments, a line and word detection process may establish a baseline for word and character shapes and separate words, if necessary. In one or more embodiments, a script recognition process may, for example in multilingual documents, identify a script, allowing an appropriate OCR algorithm to be selected. In one or more embodiments, a character isolation or “segmentation” process may separate signal characters, for example, character-based OCR algorithms. In one or more embodiments, a normalization process may normalize the aspect ratio and/or scale of image component.

With continued reference to FIG. 1, in one or more embodiments, an OCR process may include an OCR algorithm. Exemplary OCR algorithms include matrix-matching processes and/or feature extraction processes. Matrix matching may involve comparing an image to a stored glyph on a pixel-by-pixel basis. In one or more embodiments, matrix matching may also be known as “pattern matching”, “pattern recognition”, and/or “image correlation”. Matrix matching may rely on an input glyph being correctly isolated from the rest of image component. Matrix matching may also rely on a stored glyph being in a similar font and at the same scale as input glyph.

With continued reference to FIG. 1, in one or more embodiments, an OCR process may include a feature extraction process. In one or more embodiments, feature extraction may decompose a glyph into features. Exemplary nonlimiting features may include corners, edges, lines, closed loops, line direction, line intersections, and the like. In one or more embodiments, feature extraction may reduce the dimensionality of representation and may make the recognition process computationally more efficient. In one or more embodiments, extracted features can be compared with an abstract vector-like representation of a character, which might be reduced to one or more glyph prototypes. General techniques of feature detection in computer vision are applicable to this type of OCR. In one or more embodiments, machine learning process like nearest neighbor classifiers (e.g., k-nearest neighbors algorithm) can be used to compare image features with stored glyph features and choose a nearest match. OCR may employ any machine learning process described in this disclosure. Exemplary nonlimiting OCR software includes Cuneiform and Tesseract. Cuneiform is a multi-language, open-source OCR system originally developed by Cognitive Technologies of Moscow, Russia. Tesseract is a free OCR software originally developed by Hewlett-Packard of Palo Alto, California, United States.

With continued reference to FIG. 1, in one or more embodiments, OCR may employ a two-pass approach to character recognition. Second pass may include adaptive recognition and use letter shapes recognized with high confidence on a first pass to better recognize remaining letters on a second pass. In one or more embodiments, two-pass approach may be advantageous for unusual fonts or low-quality image components where visual verbal content may be distorted. Another exemplary OCR software tool includes OCRopus. The development of OCRopus is led by the German Research Center for Artificial Intelligence in Kaiserslautern, Germany. In one or more embodiments, OCR software may employ neural networks, for example, deep neural networks, as described in this disclosure below.

With continued reference to FIG. 1, in one or more embodiments, OCR may include post-processing. For example, OCR accuracy can be increased, in some cases, if output is constrained by a lexicon. A lexicon may include a list or set of words that are allowed to occur in a document. In one or more embodiments, a lexicon may include, for instance, all the words in the English language, or a more technical lexicon for a specific field. In some cases, an output stream may be a plain text stream or file of characters. In one or more embodiments, an OCR may preserve an original layout of visual verbal content. In one or more embodiments, near-neighbor analysis can make use of co-occurrence frequencies to correct errors by noting that certain words are often seen together. For example, “Washington, D.C.” is generally far more common in English than “Washington DOC”. In one or more embodiments, an OCR process may make use of a priori knowledge of grammar for a language being recognized. For example, OCR process may apply grammatical rules to help determine if a word is likely to be a verb or a noun. Distance conceptualization may be employed for recognition and classification. For example, a Levenshtein distance algorithm may be used in OCR post-processing to further optimize results. A person of ordinary skill in the art will recognize how to apply the aforementioned technologies to extract information from a digital file upon reviewing the entirety of this disclosure.

With continued reference to FIG. 1, in one or more embodiments, a computer vision module configured to perform one or more computer vision tasks such as, without limitation, object recognition, feature detection, edge/corner detection thresholding, or machine learning process may be used to recognize specific features or attributes. For the purposes of this disclosure, a “computer vision module” is a computational component designed to perform one or more computer vision, image processing, and/or modeling tasks. In one or more embodiments, computer vision module may receive from a data repository one or more digital files, such as journal article in PDF, a test result, an X-ray or CT image, an image of a prescription label, or the like, that contain medical data or features, and generate one or more labels as a function of these medical data or features. Such medical data or features may include any type of medically relevant graphical data or features. In some cases, such medical features may include the size, shape, thickness, and/or texture of one or more anatomic structures. In some cases, such medical features may include one or more graphical representations of numerical data, such as a two-dimensional plot of blood sugar level tracked over time. In one or more embodiments, to generate a plurality of labels, computer vision module may be configured to compare one or more medical features against the statistical data of the one or more medical features and attach one or more labels as a function of the comparison, consistent with details described below.

With continued reference to FIG. 1, in one or more embodiments, computer vision module may include an image processing module, wherein images may be pre-processed using the image processing module. For the purposes of this disclosure, an “image processing module” is a component designed to process digital images such as images described herein. For example, and without limitation, image processing module may be configured to compile a plurality of images of a multi-layer scan to create an integrated image. In one or more embodiments, image processing module may include a plurality of software algorithms that can analyze, manipulate, or otherwise enhance an image, such as, without limitation, a plurality of image processing techniques as described below. In one or more embodiments, computer vision module may also include hardware components such as, without limitation, one or more graphics processing units (GPUs) that can accelerate the processing of a large number of images. In one or more embodiments, computer vision module may be implemented with one or more image processing libraries such as, without limitation, OpenCV, PIL/Pillow, ImageMagick, and the like. In a nonlimiting example, in order to generate one or more labels and/or recognize one or more medical features, one or more image processing tasks, such as noise reduction, contrast enhancement, intensity normalization, image segmentation, and/or the like, may be performed by computer vision module on a plurality of images to isolate certain features or components from the rest. In one or more embodiments, one or more machine learning models may be used to perform segmentations, for example, and without limitation, a U-net (i.e., a convolution neural network containing a contracting path as an encoder and an expansive path as a decoder, wherein the encoder and the decoder forms a U-shaped structure). A person of ordinary skill in the art, upon reviewing the entirety of this disclosure, will be aware of various image processing, computer vision, and modeling tasks that may be performed by processor 104.

With continued reference to FIG. 1, in one or more embodiments, one or more functions of apparatus 100 may involve a use of image classifiers to classify images within any data described in this disclosure. For the purposes of this disclosure, an “image classifier” is a machine learning model, such as a mathematical model, neural net, or program generated by a machine learning algorithm known as a “classification algorithm”, as described in further detail below, that sort inputs of image information into categories or bins of data, outputting the categories or bins of data and/or labels associated therewith. Image classifier may be configured to output at least a datum that labels or otherwise identifies a set of images that are clustered together, found to be close under a distance metric as described below, or the like. Computing device and/or another device may generate image classifier using a classification algorithm. For the purposes of this disclosure, a classification algorithm is a process whereby computing device derives a classifier from training data. Classification may be performed using, without limitation, linear classifiers such as without limitation logistic regression and/or naive Bayes classifiers, nearest neighbor classifiers such as k-nearest neighbors classifiers, support vector machines, least squares support vector machines, Fisher's linear discriminant, quadratic classifiers, decision trees, boosted trees, random forest classifiers, learning vector quantization, and/or neural network-based classifiers. In one or more embodiments, processor 104 may use image classifier to identify a key image in any data described in this disclosure. For the purposes of this disclosure, a “key image” is an element of visual data used to identify and/or match elements to each other. In one or more embodiments, key image may include part of a medical image such as a CT scan, an MRI scan, or the like, with features that unambiguously identify the type of the medical image. Image classifier may be trained with binarized visual data that have already been classified to determine key images in any other data described in this disclosure. For the purposes of this disclosure, “binarized visual data” are visual data that are described in a binary format. For example, binarized visual data of a photo may comprise ones and zeroes, wherein the specific sequence of ones and zeros may be used to represent the photo. Binarized visual data may be used for image recognition wherein a specific sequence of ones and zeroes may indicate a product present in the image. An image classifier may be consistent with any classifier as discussed herein. An image classifier may receive input data (e.g., medical images) described in this disclosure and output a key image with the data. In one or more embodiments, image classifier may be used to compare visual data in one data set, such as medical images associated with an entity, with visual data in another data set, such as one or more medical images within a medical repository, as described below.

With continued reference to FIG. 1, processor 104 may be configured to perform feature extraction on one or more images, as described below. For the purposes of this disclosure, “feature extraction” is a process of transforming an initial data set into informative measures and values. For example, feature extraction may include a process of determining one or more geometric features of an anatomic structure. In one or more embodiments, feature extraction may be used to determine one or more spatial relationships within a drawing that may be used to uniquely identify one or more features. In one or more embodiments, processor 104 may be configured to extract one or more regions of interest, wherein the regions of interest may be used to extract one or more features using one or more feature extraction techniques.

With continued reference to FIG. 1, processor 104 may be configured to perform one or more of its functions, such as generation of plurality of content retrieval parameters 124a-n, as described above, using a feature learning algorithm. For the purposes of this disclosure, a “feature learning algorithm” is a machine learning algorithm that identifies associations between elements of data in a data set, which may include without limitation a training data set, where particular outputs and/or inputs are not specified. For instance, and without limitation, a feature learning algorithm may detect co-occurrences of elements of data, as defined above, with each other. Computing device may perform feature learning algorithm by dividing elements or sets of data into various sub-combinations of such data to create new elements of data and evaluate which elements of data tend to co-occur with which other elements. In one or more embodiments, feature learning algorithm may perform clustering of data.

With continued reference to FIG. 1, feature learning and/or clustering algorithm may be implemented, as a nonlimiting example, using a k-means clustering algorithm. For the purposes of this disclosure, a “k-means clustering algorithm” is a type of cluster analysis that partitions n observations or unclassified cluster data entries into k clusters in which each observation or unclassified cluster data entry belongs to the cluster with the nearest mean. For the purposes of this disclosure, “cluster analysis” is a process that includes grouping a set of observations or data entries in way that observations or data entries in the same group or cluster are more similar to each other than to those in other groups or clusters. Cluster analysis may be performed by various cluster models that include connectivity models such as hierarchical clustering, centroid models such as k-means, distribution models such as multivariate normal distribution, density models such as density-based spatial clustering of applications with nose (DBSCAN) and ordering points to identify the clustering structure (OPTICS), subspace models such as biclustering, group models, graph-based models such as a clique, signed graph models, neural models, and the like. Cluster analysis may include hard clustering, whereby each observation or unclassified cluster data entry belongs to a cluster or not. Cluster analysis may include soft clustering or fuzzy clustering, whereby each observation or unclassified cluster data entry belongs to each cluster to a certain degree such as for example a likelihood of belonging to a cluster; for instance, and without limitation, a fuzzy clustering algorithm may be used to identify clustering of elements of a first type or category with elements of a second type or category, and vice versa, as described below. Cluster analysis may include strict partitioning clustering, whereby each observation or unclassified cluster data entry belongs to exactly one cluster. Cluster analysis may include strict partitioning clustering with outliers, whereby observations or unclassified cluster data entries may belong to no cluster and may be considered outliers. Cluster analysis may include overlapping clustering whereby observations or unclassified cluster data entries may belong to more than one cluster. Cluster analysis may include hierarchical clustering, whereby observations or unclassified cluster data entries that belong to a child cluster also belong to a parent cluster.

With continued reference to FIG. 1, computing device may generate a k-means clustering algorithm by receiving unclassified data and outputting a definite number of classified data entry clusters, wherein the data entry clusters each contain cluster data entries. K-means algorithm may select a specific number of groups or clusters to output, identified by a variable “k”. Generating k-means clustering algorithm includes assigning inputs containing unclassified data to a “k-group” or “k-cluster” based on feature similarity. Centroids of k-groups or k-clusters may be utilized to generate classified data entry cluster. K-means clustering algorithm may select and/or be provided “k” variable by calculating k-means clustering algorithm for a range of k values and comparing results. K-means clustering algorithm may compare results across different values of k as the mean distance between cluster data entries and cluster centroid. K-means clustering algorithm may calculate mean distance to a centroid as a function of k value, and the location of where the rate of decrease starts to sharply shift, which may be utilized to select a k value. Centroids of k-groups or k-cluster include a collection of feature values which are utilized to classify data entry clusters containing cluster data entries. K-means clustering algorithm may act to identify clusters of closely related data, which may be provided with user cohort labels; this may, for instance, generate an initial set of user cohort labels from an initial set of data, and may also, upon subsequent iterations, identify new clusters to be provided new labels, to which additional data may be classified, or to which previously used data may be reclassified.

With continued reference to FIG. 1, generating a k-means clustering algorithm may include generating initial estimates for k centroids which may be randomly generated or randomly selected from unclassified data input. K centroids may be utilized to define one or more clusters. K-means clustering algorithm may assign unclassified data to one or more k-centroids based on the squared Euclidean distance by first performing a data assigned step of unclassified data. K-means clustering algorithm may assign unclassified data to its nearest centroid based on the collection of centroids ci of centroids in set C. Unclassified data may be assigned to a cluster based on argmin [symbol] dist (ci, x)², where argmin includes argument of the minimum, ci includes a collection of centroids in a set C, and dist includes standard Euclidean distance. K-means clustering module may then recompute centroids by taking a mean of all cluster data entries assigned to a centroid's cluster. This may be calculated based on ci=1/|Si|Σxi [symbol] Si^{xi}. K-means clustering algorithm may continue to repeat these calculations until a stopping criterion has been satisfied such as when cluster data entries do not change clusters, the sum of the distances have been minimized, and/or some maximum number of iterations has been reached.

With continued reference to FIG. 1, k-means clustering algorithm may be configured to calculate a degree of similarity index value. For the purposes of this disclosure, a “degree of similarity index value” is a distance measured between each data entry cluster generated by k-means clustering algorithm and a selected element. Degree of similarity index value may indicate how close a particular combination of elements is to being classified by k-means algorithm to a particular cluster. K-means clustering algorithm may evaluate the distances of the combination of elements to the k-number of clusters output by k-means clustering algorithm. Short distances between an element of data and a cluster may indicate a higher degree of similarity between the element of data and a particular cluster. Longer distances between an element and a cluster may indicate a lower degree of similarity between the element to be compared and/or clustered and a particular cluster.

With continued reference to FIG. 1, k-means clustering algorithm selects a classified data entry cluster as a function of the degree of similarity index value. In one or more embodiments, k-means clustering algorithm may select a classified data entry cluster with the smallest degree of similarity index value indicating a high degree of similarity between an element and the data entry cluster. Alternatively or additionally, k-means clustering algorithm may select a plurality of clusters having low degree of similarity index values to elements to be compared and/or clustered thereto, indicative of greater degrees of similarity. Degree of similarity index values may be compared to a threshold number indicating a minimal degree of relatedness suitable for inclusion of a set of element data in a cluster, where degree of similarity indices a-n falling under the threshold number may be included as indicative of high degrees of relatedness. The above-described illustration of feature learning using k-means clustering is included for illustrative purposes only and should not be construed as limiting potential implementation of feature learning algorithms; a person of ordinary skills in the art, upon reviewing the entirety of this disclosure, will be aware of various additional or alternative feature learning approaches, such as particle swarm optimization (PSO) and generative adversarial network (GAN) that may be used consistently with this disclosure.

With continued reference to FIG. 1, in one or more embodiments, processor 104 may use an image recognition algorithm to determine patterns within an image. In one or more embodiments, image recognition algorithm may include an edge-detection algorithm, which may detect one or more shapes defined by edges. For the purposes of this disclosure, an “edge detection algorithm” is or includes a mathematical method that identifies points in a digital image at which the image brightness changes sharply and/or has discontinuities. In one or more embodiments, such points may be organized into straight and/or curved line segments, which may be referred to as “edges”. Edge detection may be performed using any suitable edge detection algorithm, including without limitation Canny edge detection, Sobel operator edge detection, Prewitt operator edge detection, Laplacian operator edge detection, and/or differential edge detection. Edge detection may include phase congruency-based edge detection, which finds all locations of an image where all sinusoids in the frequency domain, for instance when generated using a Fourier decomposition, may have matching phases which may indicate a location of an edge.

With continued reference to FIG. 1, in one or more embodiments, one or more functions of apparatus 100, such as generation of content retrieval parameters 124a-n and prompts (including audio prompts), may be implemented by training LLM 112. For the purposes of this disclosure, a “large language model (LLM)” is a deep learning data structure that can recognize, summarize, translate, predict and/or generate text and other content based on knowledge gained from massive datasets. LLMs may be trained on large sets of data. Training sets may be drawn from diverse sets of data such as, as nonlimiting examples, scientific journal articles, medical report documents, EHRs, entity documents, business documents, inventory documentation, emails, user communications, advertising documents, newspaper articles, and the like. In some embodiments, training sets of an LLM may include information from one or more public or private databases. As a nonlimiting example, training sets may include databases associated with an entity. In some embodiments, training sets may include portions of documents associated with the electronic records correlated to examples of outputs. In one or more embodiments, LLM may include one or more architectures based on capability requirements of the LLM. Exemplary architectures may include, without limitation, Generative Pretrained Transformer (GPT), Bidirectional Encoder Representations from Transformers (BERT), Text-To-Text Transfer Transformer (T5), and the like. Architecture choice may depend on a needed capability such generative, contextual, or other specific capabilities.

With continued reference to FIG. 1, in one or more embodiments, LLM may be generally trained. For the purposes of this disclosure, a “generally trained” LLM is a LLM that is trained on a general training set comprising a variety of subject matters, data sets, and fields. In one or more embodiments, LLM may be initially generally trained. Additionally or alternatively, LLM may be specifically trained. For the purposes of this disclosure, a “specifically trained” LLM is a LLM that is trained on a specific training set, wherein the specific training set includes data including specific correlations for the LLM to learn. As a nonlimiting example, LLM may be generally trained on a general training set, then specifically trained on a specific training set. In one or more embodiments, generally training LLM may be performed using unsupervised machine learning process. In one or more embodiments, specific training of LLM may be performed using supervised machine learning process. As a nonlimiting example, specific training set may include information from a database. As a nonlimiting example, specific training set may include text related to the users such as user specific data for electronic records correlated to examples of outputs. In one or more embodiments, training one or more machine learning models may include setting the parameters of the one or more models (weights and biases) either randomly or using a pretrained model. Generally training one or more machine learning models on a large corpus of text data can provide a starting point for fine-tuning on a specific task. A model such as LLM may learn by adjusting its parameters during the training process to minimize a defined loss function, which measures the difference between predicted outputs and ground truth. Once model has been generally trained, the model may then be specifically trained to fine-tune the pretrained model on task-specific data to adapt it to the target task. Fine-tuning may involve training model with task-specific training data, adjusting the model's weights to optimize performance for the particular task. In some cases, this may include optimizing model's performance by fine-tuning hyperparameters such as learning rate, batch size, and regularization. Hyperparameter tuning may help in achieving the best performance and convergence during training. In one or more embodiments, fine-tuning pretrained model such as LLM may include fine-tuning the pretrained model using Low-Rank Adaptation (LoRA). For the purposes of this disclosure, “Low-Rank Adaptation” is a training technique for large language models that modifies a subset of parameters in the model. Low-Rank Adaptation may be configured to make the training process more computationally efficient by avoiding a need to train an entire model from scratch. In an exemplary embodiment, a subset of parameters that are updated may include parameters that are associated with a specific task or domain.

With continued reference to FIG. 1, in one or more embodiments, LLM may include and/or be produced using Generative Pretrained Transformer (GPT), GPT-2, GPT-3, GPT-4, and the like. GPT, GPT-2, GPT-3, GPT-3.5, and GPT-4 are products of Open AI Inc., of San Francisco, CA. LLM may include a text prediction-based algorithm configured to receive an article and apply a probability distribution to the words already typed in a sentence to work out the most likely word to come next in augmented articles. For example, if some words that have already been typed are “electronic health”, then it may be highly likely that the word “record” will come next. LLM may output such predictions by ranking words by likelihood or a prompt parameter. For the example given above, LLM may score “record” as the most likely, “records” as the next most likely, “profile” or “profiles” next, and the like. LLM may include an encoder component and a decoder component.

With continued reference to FIG. 1, LLM may include a transformer architecture. In some embodiments, encoder component of LLM may include transformer architecture. A “transformer architecture,” for the purposes of this disclosure is a neural network architecture that uses self-attention and positional encoding. Transformer architecture may be designed to process sequential input data, such as natural language, with applications towards tasks such as translation and text summarization. Transformer architecture may process the entire input all at once. For the purposes of this disclosure, “positional encoding” is a data processing technique that encodes the location or position of an entity in a sequence. In some embodiments, each position in the sequence may be assigned a unique representation. In some embodiments, positional encoding may include mapping each position in the sequence to a position vector. In some embodiments, trigonometric functions, such as sine and cosine, may be used to determine the values in the position vector. In some embodiments, position vectors for a plurality of positions in a sequence may be assembled into a position matrix, wherein each row of position matrix may represent a position in the sequence.

With continued reference to FIG. 1, LLM and/or transformer architecture may include an attention mechanism. For the purposes of this disclosure, an “attention mechanism” is a part of a neural architecture that enables a system to dynamically quantify relevant features of the input data. In the case of natural language processing, input data may be a sequence of textual elements. It may be applied directly to the raw input or to its higher-level representation.

With continued reference to FIG. 1, attention mechanism may represent an improvement over a limitation of an encoder-decoder model. An encoder-decider model encodes an input sequence to one fixed length vector from which the output is decoded at each time step. This issue may be seen as a problem when decoding long sequences because it may make it difficult for the neural network to cope with long sentences, such as those that are longer than the sentences in the training corpus. Applying attention mechanism, LLM may predict next word by searching for a set of positions in a source sentence where the most relevant information is concentrated. LLM may then predict next word based on context vectors associated with these source positions and all the previously generated target words, such as textual data of a dictionary correlated to a prompt in a training data set. For the purposes of this disclosure, “context vectors” are fixed-length vector representations useful for document retrieval and word sense disambiguation.

With continued reference to FIG. 1, attention mechanism may include, without limitation, generalized attention, self-attention, multi-head attention, additive attention, global attention, and the like. In generalized attention, when a sequence of words or an image is fed to LLM, it may verify each element of input sequence and compare it against the output sequence. Each iteration may involve the mechanism's encoder capturing input sequence and comparing it with each element of the decoder's sequence. From the comparison scores, attention mechanism may then select the words or parts of image that it needs to pay attention to. In self-attention, LLM may pick up particular parts at different positions in input sequence and over time compute an initial composition of output sequence. In multi-head attention, LLM may include a transformer model of an attention mechanism. Attention mechanisms, as described above, may provide context for any position in input sequence. For example, if the input data is a natural-language sentence, the transformer does not have to process one word at a time. In multi-head attention, computations by LLM may be repeated over several iterations, and each computation may form parallel layers known as attention heads. Each separate head may independently pass input sequence and corresponding output sequence element through separate head. A final attention score may be produced by combining attention scores at each head so that every nuance of input sequence is taken into consideration. In additive attention (Bahdanau attention mechanism), LLM may make use of attention alignment scores based on a number of factors. Alignment scores may be calculated at different points in neural network, and/or at different stages represented by discrete neural networks. Source or input sequence words are correlated with target or output sequence words but not to an exact degree. This correlation may take into account all hidden states and the final alignment score is the summation of a matrix of alignment scores. In global attention (Luong mechanism), in situations where neural machine translations are required, LLM may either attend to all source words or predict the target sentence, thereby attending to a smaller subset of words.

With continued reference to FIG. 1, multi-headed attention in encoder may apply a specific attention mechanism called self-attention. Self-attention allows models such as LLM or components thereof to associate each word in input, to other words. As a nonlimiting example, LLM may learn to associate the word “you”, with “how” and “are”. It's also possible that LLM learns that words structured in this pattern are typically a question and to respond appropriately. In one or more embodiments, to achieve self-attention, input may be fed into three distinct and fully connected neural network layers to create query, key, and value vectors. Query, key, and value vectors may be fed through a linear layer; then, the query and key vectors may be multiplied using dot product matrix multiplication in order to produce a score matrix. Score matrix may determine the amount of focus for a word that should be put on other words (thus, each word may be a score that corresponds to other words in the time-step). The values in score matrix may be scaled down. As a nonlimiting example, score matrix may be divided by the square root of the dimension of the query and key vectors. In one or more embodiments, a softmax of the scaled scores in score matrix may be taken. The output of this softmax function may be called attention weights. Attention weights may be multiplied by your value vector to obtain an output vector, wherein the output vector may then be fed through a final linear layer.

With continued reference to FIG. 1, in order to use self-attention in a multi-headed attention computation, query, key, and value may be split into N vectors before applying self-attention. Each self-attention process may be called a “head”. Each head may produce an output vector and each output vector from each head may be concatenated into a single vector. This single vector may then be fed through final linear layer discussed above. In theory, each head can learn something different from input, therefore giving the encoder model more representation power.

With continued reference to FIG. 1, encoder of transformer may include a residual connection. Residual connection may include adding the output from multi-headed attention to the positional input embedding. In one or more embodiments, an output from residual connection may go through a layer normalization. In one or more embodiments, a normalized residual output may be projected through a pointwise feed-forward network for further processing. Pointwise feed-forward network may include a couple of linear layers with a ReLU activation in between. Output may then be added to an input of the pointwise feed-forward network and further normalized.

With continued reference to FIG. 1, transformer architecture may include a decoder. Decoder may a multi-headed attention layer, a pointwise feed-forward layer, one or more residual connections, and layer normalization (particularly after each sub-layer), as discussed in more detail above. In one or more embodiments, decoder may include two multi-headed attention layers. In one or more embodiments, decoder may be autoregressive. For the purposes of this disclosure, “autoregressive” means that the decoder takes in a list of previous outputs as inputs along with encoder outputs containing attention information from the input.

With continued reference to FIG. 1, in one or more embodiments, input to decoder may go through an embedding layer and positional encoding layer to obtain positional embeddings. Decoder may include a first multi-headed attention layer, wherein the first multi-headed attention layer may receive positional embeddings.

With continued reference to FIG. 1, first multi-headed attention layer may be configured to not condition to future tokens. As a nonlimiting example, when computing attention scores on the word “am”, decoder should not have access to the word “fine” in “I am fine”, because that word is a future word that was generated after. The word “am” should only have access to itself and the words before it. In one or more embodiments, this may be accomplished by implementing a look-ahead mask. Look ahead mask is a matrix of the same dimensions as a scaled attention score matrix that is filled with “Os” and negative infinities. For example, the top right triangle portion of look-ahead mask may be filled with negative infinities. Look-ahead mask may be added to scaled attention score matrix to obtain a masked score matrix. Masked score matrix may include scaled attention scores in the lower-left triangle of the matrix and negative infinities in the upper-right triangle of the matrix. Then, when a softmax of this matrix is taken, negative infinities will be zeroed out; this leaves zero attention scores for “future tokens”.

With continued reference to FIG. 1, second multi-headed attention layer may use encoder outputs as queries and keys and outputs from the first multi-headed attention layer as values. This process matches encoder's input to the decoder's input, allowing the decoder to decide which encoder input is relevant to put a focus on. An output from second multi-headed attention layer may be fed through a pointwise feedforward layer for further processing.

With continued reference to FIG. 1, an output of the pointwise feedforward layer may be fed through a final linear layer. This final linear layer may act as a classifier. This classifier may be as big as the number of classes that you have. For example, if you have 10,000 classes for 10,000 words, output of that classifier will be of size 10,000. Output of this classifier may be fed into a softmax layer which may serve to produce probability scores between zero and one. An index may be taken of the highest probability score in order to determine a predicted word.

With continued reference to FIG. 1, decoder may take this output and add it to decoder inputs. Decoder may continue decoding until a token is predicted. Decoder may stop decoding once it predicts an end token.

With continued reference to FIG. 1, in one or more embodiments, decoder may be stacked N layers high, with each layer taking in inputs from encoder and layers before it. Stacking layers may allow LLM to learn to extract and focus on different combinations of attention from its attention heads.

With continued reference to FIG. 1, LLM may receive an input. Input may include a string of one or more characters. Inputs may additionally include unstructured data. For example, input may include one or more words, a sentence, a paragraph, a thought, a query, and the like. For the purposes of this disclosure, a “query” is a string of characters that poses a question. In one or more embodiments, input may be received from a user device. User device may be any computing device that is used by a user. As nonlimiting examples, user device may include desktops, laptops, smartphones, tablets, and the like. In one or more embodiments, input may include any set of data associated with training and/or using LLM. As a nonlimiting example, input may be a prompt such as “what potential medical issues can be found in this patient's electronic health records?”

With continued reference to FIG. 1, LLM may generate at least one annotation as output. At least one annotation may be any annotation as described herein. In one or more embodiments, LLM may include multiple sets of transformer architecture as described above. Output may include a textual output. For the purposes of this disclosure, “textual output” is an output comprising a string of one or more characters. Textual output may include, for example, a plurality of annotations for unstructured data. In one or more embodiments, textual output may include a phrase or sentence identifying the status of a user query. In one or more embodiments, textual output may include a sentence or plurality of sentences describing a response to user query. As a nonlimiting example, this may include restrictions, timing, advice, dangers, benefits, and the like.

With continued reference to FIG. 1, processor 104 is configured to receive, from a first entity 128, an input data structure 132 as a function of the plurality of content retrieval parameters 124a-n. For the purposes of this disclosure, an “entity” is an individual (i.e., a natural person), a group of individuals, a corporate or organization, a department or division within a corporate or organization, or otherwise any subject or party capable of participating in one or more functions of apparatus 100 described in this disclosure. As a nonlimiting example, first entity 128 may include a patient, his/her/their primary care physician, one or more of his/her/their representatives, and/or the like. For the purposes of this disclosure, an “input” is an organized collection of medically relevant data elements provided by an entity in response to plurality of content retrieval parameters 124a-n. For the purposes of this disclosure, an “input data structure” is a data structure configured to organize one or more data elements. Input data structure 132 may include any type of input or combination of inputs consistent with details described above regarding training examples 120 and may implement any type of data structure described in this disclosure without limitation. As nonlimiting examples, input data structure may include textual data such as a nurse or doctor's notes archived in EHRs, audio data such as voice inputs describing the recent medical history of the first entity 128, or graphical data such as an image of a prescription bottle that contains the name and dosage of a medication.

With continued reference to FIG. 1, in one or more embodiments, accepting input data structure 132 may include retrieving, from a data repository 136, a first input element pertaining to first entity 128, and receiving, from the first entity 128, a second input element as a function of the first input element. Data repository 136 may include or be implemented in accordance with any type of database or the like as described in this disclosure. In one or more embodiments, input data structure 132 may include at least a digital file, and accordingly, at least a content retrieval data structure 116 may include LLM 112 configured to extract textual data from the at least a digital file, consistent with details described above. In some cases, receiving input data structure 132 may further include validating first input element as a function of second input element, and updating the input data structure 132 as a function of an outcome of the validation.

With continued reference to FIG. 1, in one or more embodiments, receiving second input element may include generating, using at least a content retrieval data structure 116, at least a prompt 140 as a function of first input element and plurality of content retrieval parameters 124a-n. For the purposes of this disclosure, a “prompt” is a query used by one party to collect one or more replies from another party. Prompt 140 may include and/or be implemented in accordance with any possible types of prompts. As a nonlimiting example, prompt 140 may include one or more surveys, questionnaires, and/or URLs transmitted to first entity 128 by email or text message, as described below. Accordingly, receiving second input element may further include receiving from first entity 128, secondary input data in response to at least a prompt 140 and updating input data structure 132 as a function of the secondary input data. As a nonlimiting example, apparatus 100 may identify from data repository 136 at least a first input element indicating that first entity 128, John Doe, i) is scheduled for a cholecystectomy in four weeks and ii) is currently prescribed with several types of medications including 1,000 mg of metformin a day that treat diabetes. Accordingly, apparatus 100 may send prompt 140 to John Doe to validate such information on file and collect second input element including additional information associated with one or more related issues, such as i) whether he has checked into an emergency room or been hospitalized recently, ii) how his diabetes has been managed in the past three months, iii) whether he's actively taking metformin, and if so, at what dosage, iv) whether he has symptoms of asthma or chronic obstructive pulmonary disease (COPD), and v) whether he has certain heart conditions or risk factors associated with potential strokes, among others. Information gathered therefrom may then be recorded by apparatus 100 to update input data structure 132.

With continued reference to FIG. 1, in one or more embodiments, apparatus 100 may include a sound capturing device communicatively connected to processor 104. For the purposes of this disclosure, a “sound capturing device” is a device capable of capturing audio information by isolating it from its background noise. In some cases, sound capturing device may be configured to establish a secure communication channel between first entity 128 and a second entity 144 and transmit audio data (e.g., prompts 140, secondary input data, or the like) between the first entity 128 and the second entity 144. As a nonlimiting example, sound capturing device may include a microphone integrated within a desktop, a laptop, a tablet, or a cellphone. For the purposes of this disclosure, a “microphone” is a transducer configured to transduce pressure change phenomenon to a signal, for instance a signal representative of a parameter associated with the phenomenon. Microphone, according to some embodiments, may include a transducer configured to convert sound into electrical signal. Exemplary nonlimiting microphones include dynamic microphones (which may include a coil of wire suspended in a magnetic field), condenser microphones (which may include a vibrating diaphragm condensing plate), and a contact (or conductance) microphone (which may include piezoelectric crystal material). Microphone may include any microphone for transducing pressure changes, as described above; therefore, microphone may include any variety of microphone, including any of: condenser microphones, electret microphones, dynamic microphones, ribbon microphones, carbon microphones, piezoelectric microphones, fiber-optic microphones, laser microphones, liquid microphones, microelectromechanical systems (MEMS) microphones, and/or a speaker microphone. In such embodiments, receiving second input element may further include synthetizing, using a speech synthesis algorithm 148, an audio prompt 140 as a function of first input element and the plurality of content retrieval parameters 124a-n, and capturing, using sound capturing device, audio secondary input data from first entity in response to the audio prompt 140. Accordingly, receiving second input element may further include transcribing audio secondary input data into textual secondary input data using at least a content retrieval data structure 116 and updating input data structure 132 as a function of the textual secondary input data. For the purposes of this disclosure, a “text-to-speech” or “speech synthesis” algorithm is a machine learning algorithm configured to synthesize language text into speech, capturing nuances such as without limitation dialects, accents, pausing, and intonations. In some cases, an LLM may first be used to generate prompts with complete sentences or paragraphs, wherein speech synthesis algorithm 148 may further convert such prompts into audio data. In some cases, speech synthesis algorithm 148 may be implemented as a trained text-to-speech machine learning model. Specifically, training such text-to-speech machine learning model may include receiving speech synthesis training data comprising a plurality of training texts as inputs and a plurality of training audio data as outputs, training the text-to-speech machine learning model 148 by correlating the plurality of training texts with the plurality of training audio data, and synthesizing audio prompt 140 using the text-to-speech machine learning model 148. Implementation of this machine learning model may be consistent with any type of machine learning model or algorithm described in this disclosure. In one or more embodiments, speech synthesis training data may include data specifically synthesized for training purposes using one or more generative models, as described in this disclosure. In one or more embodiments, one or more input data structures 132 from previous sessions may be incorporated into speech synthesis training data upon validation. In one or more embodiments, speech synthesis training data may be retrieved from one or more databases and/or other repositories of similar nature or be supplied as one or more inputs from one or more entities. In one or more embodiments, at least a portion of speech synthesis training data may be added, deleted, replaced, or otherwise updated as a function of one or more inputs from one or more entities. A person of ordinary skill in the art, upon reviewing the entirety of this disclosure, will be able to recognize suitable means to implement text-to-speech algorithm in apparatus 100.

With continued reference to FIG. 1, in one or more embodiments, apparatus 100 may include or be communicatively connected to an image capturing device configured to capture one or more images. For the purposes of this disclosure, an “image capturing device” is a device capable of recording a digital representation of an object. Image capture device may include any type of image capture device accessible to a person of ordinary skill in the art, and/or deemed suitable by a person of ordinary skill in the art upon reviewing the entirety of this disclosure. In one or more embodiments, image capturing device may also function as a display device, as described below. In one or more embodiments, image capturing device and display device may be integrated into a single device. In one or more embodiments, image capture device may include a camera. For the purposes of this disclosure, a “camera” is a single device or an assembly of multiple devices configured to detect at least one type of electromagnetic radiation and generate a graphical representation therefrom. As nonlimiting examples, camera may detect visible light, infrared light, ultraviolet light, or X-ray. In one or more embodiments, camera may include one or more optics; nonlimiting examples of optics include spherical lenses, aspherical lenses, reflectors, polarizers, filters, windows, aperture stops, and the like. In one or more embodiments, camera may include an image sensor. Exemplary image sensors include digital image sensors, such as without limitation charge-coupled device (CCD) sensors and complimentary metal-oxide-semiconductor (CMOS) sensors. As a nonlimiting example, camera may include a remote camera device communicatively connected to a computing device, such as a portable camera connected to a desktop or laptop computer through either a cord or wireless connection. As a nonlimiting example, camera may include a camera integrated within a computing device, such as a built-in camera of a laptop computer. As another nonlimiting example, camera may include a camera integrated within a remote and/or portable device, such as a built-in camera of a smartphone or a tablet. For the purposes of this disclosure, an “image” is a visual representation of data. In some embodiments, image may be product of image capture device. In some embodiments, image may contain digital information representing at least a physical scene, space, and/or object. In one or more embodiments, image may be an optical image, such as without limitation an image of an object generated by at least an optic. In some cases, image may be a digital representation of another image, such as a digital image of a printed photograph or the like captured using a built-in camera of a smartphone. Alternatively, image may comprise a plurality of images arranged in sequence as a function of time, such as one or more videos. In some embodiments, image may include a digital image. Digital image may be in a format such as jpeg, png, pdf, btmp, and the like. As a nonlimiting example, first entity 128 may use image capturing device, such as a built-in camera of a cell phone, to capture and upload an image of a prescription label, wherein apparatus 100 may be configured to extract information from the prescription label, e.g., using ORR, to populate or update input data structure 132.

With continued reference to FIG. 1, processor 104 may be configured to implement a multichannel protocol, such as voice, text, email, and one or more combinations thereof, among others, in order to communicate with one or more entities described in this disclosure. As a nonlimiting example, apparatus 100 may implement an AI-based patient concierge to share updates, gather intakes, answer questions, and coordinate appointments before and after a surgery. As another nonlimiting example, apparatus 100 may accept from a patient a picture of a medication, extract information therefrom, and incorporate the extracted information as an input for determining downstream pathways.

With continued reference to FIG. 1, processor 104 is further configured to populate, using input data structure 132, a content queue 152 comprising a plurality of content elements 156a-n. For the purposes of this disclosure, a “content queue” is a list of data elements generated as a function of one or more elements of input data structure 132, as described above, and arranged in a pre-determined order for subsequent steps. In some ways, content queue 152 may resemble a to-do list of a personal assistant acting on behalf of first entity 128. For the purposes of this disclosure, a “queue” is a collection of entities that are maintained in a sequence and can be modified by the addition of entities at one end of the sequence and the removal of entities from the other end of the sequence. By convention, the end of the sequence at which elements are added is called the back, tail, or rear of the queue, and the end at which elements are removed is called the head or front of the queue, analogously to the words used when people line up to wait for goods or services. In other words, a queue represents a “first-in-first-out” data structure wherein the first element added to the queue will be the first one to be removed. For the purposes of this disclosure, a “content element” is an item relevant to one or more content retrieval parameters 124a-n, one or more features of input data structure 132, and/or one or more elements within input data structure 132 that need to be addressed, communicated, validated, updated, and/or the like. As a nonlimiting example, plurality of content elements 156a-n may include a plurality of reminders, such as medications to start or stop using, food or drink to start/stop consuming, expected time and location of check-in, as well as other similar preparatory tasks relevant for a medical procedure. As another nonlimiting example, plurality of content elements 156a-n may include a plurality of scheduled phone calls to make with a plurality of entities, a plurality of electronic documents to transmit for surgeons, nurses, anesthesiologists, nutritionists, and/or administrative support staff to review, confirm, sign, and/or approve, a plurality of orders or prescriptions to submit for back-end processing, among others. As a nonlimiting example, apparatus 100 may implement an AI-driven document management module to perform an automated assessment of faxed, scanned, or emailed records regarding each patient. As another nonlimiting example, apparatus 100 may be configured to automate nurse follow-ups from pre-admission testing phone calls, including generation of EHR notes, management of medical records requests, and communication of instructions to patients, among others. In some cases, plurality of content elements 156a-n within content queue 152 may be organized in a sequential order, e.g., an order in which actions may be taken or an order in which deadlines are to be met. In some cases, plurality of content elements 156a-n within content queue 152 may be arranged as a function of one or more factors of importance. In some cases, plurality of content elements 156a-n within content queue 152 may be rearranged as a function of one or more inputs by one or more entities. In some cases, one or more content elements 156a-n within content queue 152 may be added, deleted, replaced, or otherwise updated as a function of one or more inputs by one or more entities.

With continued reference to FIG. 1, in one or more embodiments, processor 104 may be configured to identify from input data structure 132 one or more risk factors associated with first entity 128 and assign one or more numerical, categorical, and/or descriptive risk score such as 9/10, 80/100, or “severe”. Accordingly, processor 104 may be configured to suggest one or more means of optimizations such as treatment plans, diets, referrals of specialists, among others. In some cases, apparatus 100 may offer continuous patient & surgical risk stratification using AI and/or machine learning. In some cases, apparatus 100 may be used to generate patient-specific care coordination plans and pathways. As a nonlimiting example, risk stratification and create pathways may be determined as a function of data pertaining to first entity 128. For a high-risk patient, a doctor, medical professional, or support staff member may be scheduled to give a call one week before a scheduled procedure. In some cases, apparatus 100 may offer systematic identification of patient optimization opportunities and automated coordination of care. Such use may be relevant to patients diagnosed with medical conditions such as diabetes, anemia, among others, that potentially complicate a medical procedure to be performed. As a nonlimiting example, apparatus 100 may remind a patient to see a cardiologist before the surgery. Additional details will be provided below in this disclosure.

With continued reference to FIG. 1, apparatus 100 may perform one or more of its functions using a web crawler. For the purposes of this disclosure, a “web crawler” is a program that systematically browses the internet for web indexing. Web crawler may be seeded with platform URLs, wherein the web crawler may then visit the next related URL, retrieve the content, index the content, and/or measure the relevance of the content to the topic of interest. In one or more embodiments, computing device may generate web crawler to compile and/or generate a plurality of matched results. Web crawler may be seeded and/or trained with a reputable website, such as a relevant governmental website. Web crawler may be generated by computing device. In one or more embodiments, web crawler may be configured to generate a web query, wherein the web query may include information contained in input data structure 132, as described below. As a nonlimiting example, apparatus 100 may submit a plurality of websites for web crawler to search to extract any data that match one or more elements within input data structure 132. In one or more embodiments, web crawler may allow for a data store, such as data repository 136, to be populated with laws, regulations, ordinances, and the like provided by the local authorities. In one or more embodiments, some requirements may be categorized based on at least a geographic location, wherein web crawler may be configured to retrieve requirements based on the at least a geographic location, consistent with details described in this disclosure. As a nonlimiting example, training examples 120 may be retrieved from, synthesized, and/or dynamically updated using online resources using a web crawler. As another nonlimiting example, the contact information, address, business hours, insurance policies, and the like pertaining to a hospital or medical practitioner may be retrieved using a web crawler for apparatus 100 to perform subsequent tasks, as described below.

With continued reference to FIG. 1, processor 104 is further configured to query at least a second entity 144 using at least a content element 156a-n of plurality of content elements 156a-n. Second entity 144 may include any entity that is different from first entity 128, such as without limitation one or more surgeons, nurses, anesthesiologists, nutritionists, and/or administrative support staff, that may be capable of providing relevant information regarding one or more content elements 156a-n. Query may be communicated using any means consistent with details described above regarding receipt of input data structure 132 and/or otherwise deemed suitable by a person of ordinary skill in the art upon reviewing the entirety of this disclosure. Exemplary means of communication may include without limitation, a fax, an email, or a text message containing one or more documents/electronic documents to sign, a prescription to approve, a survey or questionnaire to complete, and/or a URL for uploading files or documents. Additionally, and/or alternatively, exemplary means of communication may include automated phone calls using one or more synthetic audio prompts, consistent with details described above.

With continued reference to FIG. 1, in one or more embodiments, processor 104 may be configured to apply a geofence when performing one or more of its functions, such as querying one or more second entity 144. In some cases, certain inclusion/exclusion criteria may be applied to a plurality of second entities to selectively isolate the portion thereof within a geofence. As a nonlimiting example, a medical procedure may require that a practitioner be registered within a certain state, and apparatus 100 may apply a geofence to reflect such requirement when selecting a practitioner. For purposes of this disclosure, a “geofence” or “geofenced area” is a virtual perimeter or boundary defined by geographic coordinates in a digital mapping system. Geographical coordinates may include a radius from a geographical point, proximity to a landmark, zip codes, area codes, longitude and latitude, cities, states, countries, counties, travel time, and/or the like. Geofence may be generated as a radius around a point or location (e.g., a detected location of first entity 128 based on an associated IP address) or using arbitrary borders drawn by user (e.g., the borders a neighborhood). In some embodiments, the point or location may be selected by an entity, such as first entity 128, through one or more secondary inputs, which may include, as nonlimiting examples, tapping on a screen, inputting an address, inputting coordinates, and/or the like. Geofences may be generated to match a predetermined set of boundaries such as neighborhoods, school zones, zip codes, county, state, and city limits, area codes, voting districts, geographic regions, streets, rivers, other landmarks, and/or the like. In one or more embodiments, geofences may be generated as a function of input data structure 132 using one or more addresses detected therein. Geofences may be used in location-based services and applications to trigger specific actions or events when a mobile device or GPS-enabled object enters, exits, or remains within a designated area. In one or more embodiments, user interface on one or more remote devices may be updated as a function of an entity's physical presence within geofenced area. Geofence may include a proximity geofence. For the purposes of this disclosure, a “proximity geofence” is a virtual boundary or area defined by geographical coordinates which is used to trigger specific actions or events; this may include events such as when a mobile device or object enters or exits what is outlined by the proximity-based geofence. Proximity geofences are used to trigger events when a device or location is within or near the vicinity of a specific location; they are often used for location-based marketing and notifications. This may be used to let an entity know when they are nearing the boundary for one or more geofenced areas. For example, processor 104 may be configured to first entity 128 a notification stating that the first entity 128 is within 1000 ft of a hospital or emergency room. In one or more additional embodiments, geofence may include a dynamic geofence. Dynamic geofences can change in real-time based on variables like first entity's location, device data, environmental factors, user preferences, and/or the like. This allows for adaptive and context-aware geofencing applications. In one or more embodiments, geofence may include a time-based geofence. In addition to geographical boundaries, time-based geofences trigger events or notifications based on specific time intervals or schedules. For example, first entity 128 may have to be physically located within geofenced area for at least 1 minute in order to receive notifications about available medical resources nearby.

With continued reference to FIG. 1, processor 104 is further configured to receive from at least a second entity 144, at least a query response 160a-n as a function of query, and update content queue 152 as a function of the at least a query response 160a-n. For the purposes of this disclosure, a “query response” is a collection of data or information prepared by a party in response to a request made by another party. In one or more embodiments, updating content queue may include confirming, by a third entity 164, at least a content element 156a-n of content queue 152 and updating the content queue 152 as a function of an outcome of the confirmation. Third entity 164 may include any entity different from first entity 128 and second entity 144. As a nonlimiting example, third entity may include one or more team members in charge of maintaining apparatus 100 or one or more pre-admission testing nurses. Successful receipt of query response 160a-n and/or a positive confirmation associated thereto may be indicated using a first indication, such a green token, as described below. However, in some cases, updating content queue 152 may further include identifying at least a content disagreement 168 by comparing at least a query response 160a-n against at least an input data structure 132, annotating at least a content element 156a-n as a function of the at least a content disagreement 168, and updating the at least a content element 156a-n by resolving, at third entity 164, the at least a content disagreement 168. For the purposes of this disclosure, a “content disagreement” is a case or scenario where the information contained in a first data set is not matching its corresponding information in a second data set. In some cases, at least a query response 160a-n may include at least a digital file. Accordingly, updating the content queue 152 may include extracting at least a query response element from at least a digital file using at least a content retrieval data structure 116 and updating at least a content element 156a-n of plurality of content elements 156a-n using the at least a query response element. Such tasks may be completed or assisted by using LLM 112. For the purposes of this disclosure, a “query response element” is an item within query response 160a-n that addresses or corresponds to one or more content elements 156a-n. As a nonlimiting example, a query response element may include the address of a medical facility, the contact information of a physician, the name and/or dosage of a prescribed medication, or the like. Continuing the same nonlimiting example described above, apparatus 100 may identify from input data structure 132 that first entity 128, Jon Doc, is scheduled for cholecystectomy that requires full anesthesia, yet query response 160a-n containing a prescription submitted by second entity 144, such as an anesthesiologist, includes only an order of local anesthesia. Apparatus 100 may flag such disagreement by generating a second indication that is different from first indication, such as a red token instead of a green token. Third entity may then be prompted to verify at least a content disagreement 168 as a function of the annotation and update at least a content element 156a-n as a function of the verification. Continuing the same nonlimiting example, a team member maintaining apparatus 100 may verify that John Doe's surgery does require full anesthesia and submit a command to apparatus 100 to generate a follow-up query requesting the anesthesiologist to resubmit a correct prescription for full anesthesia; once such content disagreement 168 is resolved, content element 156a-n regarding anesthesia may be updated by removing the annotation, e.g., by replacing second indication (red token) with first indication (green token), either automatically or manually.

With continued reference to FIG. 1, processor 104 is further configured to generate a recommended course of action 172 as a function of content queue 152. For the purposes of this disclosure, a “recommended course of action” is a single action or a series of actions to be performed according to content elements 156a-n within content queue 152. It may refer to any format pertaining to a delivery of information, a performance of activity, or the like, that is deemed relevant to apparatus 100 by a person of ordinary skill in the art, upon reviewing the entirety of this disclosure. As a nonlimiting example, recommended course of action may include a structured collection of medically relevant information or instructions that first entity 128 is expected to be aware of or comply with. As another nonlimiting example, recommended course of action 172 may include food, drink, and/or substance to avoid beyond a certain timestamp. As another nonlimiting example, recommended course of action 172 may include a follow-up phone-call to be made, an appointment to be scheduled, a manual review to be performed, a schedule to be revised or updated, or the like. In some cases, recommended course of action 172 may be synthesized and/or communicated using one or more prompts generated using one or more key words from updated content queue 152, through one or more machine learning models (e.g., generative models such as LLM 112 described above).

With continued reference to FIG. 1, accordingly, processor 104 is further configured to perform recommended course of action 172. In some cases, performing recommended course of action 172 may include displaying the recommended course of action 172 using a display device, as described below in this disclosure. In some cases, performing recommended course of action 172 may include automatically communicating with one or more computing devices to initiate one or more components of the recommended course of action 172. Such computing systems may include apparatus 100, one or more remote or mobile devices, and/or the like that may include or be communicatively connected to data repository 136, database, EHRs, web crawlers, or another collection of data of similar nature. In some cases, performing recommended course of action 172 may include placing one or more automatic phone calls, sending one or more automatic text messages, emails, or voicemails, or the like. In some cases, performing recommended course of action 172 may include adding, deleting, modifying, or otherwise updating one or more data elements, such as creating, cancelling, rescheduling, and/or updating an appointment. Continuing the same nonlimiting example described above in this disclosure, apparatus 100 may display to John Doe, using text message, email, automated phone call, or the like, that he is expected to stop taking metformin 2 days prior to his scheduled surgery. Additionally, and/or alternatively, recommended course of action may include one or more reminders regarding check-in time and/or check-in location. In one or more embodiments, generating and/or displaying recommended course of action 172 may include ranking plurality of content elements 156a-n as a function of one or more pre-determined criteria, such as temporal sequence or level of importance, consistent with details described above. Accordingly, processor 104 may be configured to generate and/or display recommended course of action 172 based on the rank of plurality of content elements 156a-n. Continuing the same nonlimiting example, apparatus 100 may send to John Doe a plurality of reminders by following a structured timeline such as two weeks, one week, and one day prior to his scheduled cholecystectomy.

With continued reference to FIG. 1, in one or more embodiments, apparatus 100 may be configured to be fully automated without any need for human intervention. Such embodiments may be implemented for relatively straightforward tasks such as receiving input data structure 132. In one or more embodiments, apparatus 100 may delegate a certain portion of its tasks for manual processing based on one or more preset criteria (i.e., pre-determined human-in-the loop embodiments). In one or more embodiments, apparatus 100 may be configured to trigger human intervention when needed (i.e., reactionary human-in-the loop embodiments). Such embodiments may be implemented when one or more tasks performed by apparatus 100 become abnormally complex or inefficient in nature, or one or more entities start to express concern or dissatisfaction. As a nonlimiting example, apparatus 100 may detect that a task is taking twice the usual time under automated mode and redirect the task to be completed by a customer service representative. As another nonlimiting example, one or more entities may simply state “I would like to speak with a customer service representative” during an automated phone call to direct apparatus 100 to switch from an automated mode to a manual mode. In some cases, apparatus 100 may implement a verification module, such as a rule engine, that enforces one or more rules and/or generates one or more prompts when one or more rules are not met. For the purposes of this disclosure, a “verification module” is a module that contains one or more models or algorithms configured to perform one or more verification tasks. For the purposes of this disclosure, “verification” is a process of ensuring that an item that is being verified complies with certain constraints. As nonlimiting examples, such constraints may include system requirements, regulations, and/or the like. For the purposes of this disclosure, a “rule engine” is a computational system that uses a set of predefined rules or criteria to make decisions based on input data, wherein each rule within the set of predefined rules is a specific criterion or condition that dictates what actions apparatus 100 should perform and/or if human intervention may be needed. In some cases, rule engine may be configured to mark one or more input data elements in input data structure 132 as mandatory (i.e., cannot be left as blank). In some cases, such set of predefined rules may be based on preferences, rules, and/or requirements set forth by one party (such as first entities 128, one or more second entities 144, and/or third entity 164) that limits the options available for another party (such as first entities 128, one or more second entities 144, and/or third entity 164). As a nonlimiting example, rule engine may include a rule stating that “a manual review is needed when more than three content disagreements 168 are identified”. As another nonlimiting example, rule engine may include another rule stating that “first entity 128 must update input data structure 132 with new medical exam results when previous results were collected more than six months prior to scheduled surgery”. As another nonlimiting example, rule engine may include another rule stating that “second entity 144 does not accept out-of-network patients”. Accordingly, verification module may be configured to use such rule engine to flag one or more items, such as one or more content elements 156, when one or more mandatory input elements of input data structure 132 are missing and/or or when one or more descriptors that describe content disagreement 168 exceed a threshold value. As a nonlimiting example, third entity 164, such as a pre-admission testing nurse, may reach out to first entity 128 to follow up as the date of surgery approaches to resolve any outstanding issues such as missing health insurance information, outdated/inconsistent medical exam results, or the like. Additional details are provided below in several nonlimiting examples. A person of ordinary skill in the art will be able to recognize proper means to implement verification modules, rule engines, and/or any element related thereto upon reviewing the entirety of this disclosure.

With continued reference to FIG. 1, it is worth noting that, in some cases, the assignment of first entity 128, second entity 144, or third entity 164 may be arbitrary, as the three may be interchangeable with one another for apparatus 100. In other words, the assignment of first entity 128, second entity 144, or third entity 164 may depend on which of the three entities that apparatus 100 is associated with and configured to represent. As a nonlimiting example, a patient may be represented by a first agent, and a surgeon may be represented by a second agent, and a pre-admission testing nurse may be represented by a third agent, wherein each agent is essentially based on a distinct content retrieval data structure 116. Accordingly, one or more functions of apparatus 100 may be performed by transmission or exchange of data between one agent and another, without human intervention. As a nonlimiting example, scheduling of an appointment may be completed by an automated phone call between two agents representing a patient/pre-admission testing nurse and a surgeon, respectively.

With continued reference to FIG. 1, in one or more embodiments, displaying recommended course of action 172 may include receiving at least a supplemental input from one or more of first entity 128 and second entity 144 and modifying recommended course of action 172 based on the at least a supplemental input. Continuing the same nonlimiting example, John Doe may report his recent change in medication, such as an increased dosage of metformin from 1,000 mg to 1,500 mg per day, and as a result, apparatus 100 may modify recommended course of action by suggesting John Doe stop taking metformin 4 days prior to his scheduled surgery instead of 2 days, allowing for sufficient time for metformin to be cleared from his system. As another nonlimiting example, John Doe's surgeon, Jane Smith, may report an unexpected schedule change, and as a result, apparatus 100 may modify recommended course of action by proposing to John Doe an alternative day/time for his cholecystectomy. As another nonlimiting example, the hospital in which John Doe is expected to have his cholecystectomy may report an unexpected change in the location of operation, and as a result, apparatus 100 may modify recommended course of action by proposing to John Doe an updated floor/room number for his cholecystectomy.

With continued reference to FIG. 1, in one or more embodiments, apparatus 100 may implement one or more feedback loops to use one or more of its outputs as inputs in order to perform or improve one or more of its functions. As a nonlimiting example, audio recordings and textual transcripts from previous sessions may be incorporated into training examples 120 upon receiving a consent from first entity 128 and/or second entity 144.

With continued reference to FIG. 1, in one or more embodiments, apparatus 100 may include or be communicatively connected to a display device 176, wherein the display device 176 is configured to display recommended course of action 172. For the purposes of this disclosure, a “display device” is a device configured to show visual information. In some cases, display device 176 may include a liquid crystal display (LCD), a cathode ray tube (CRT), a plasma display, a light emitting diode (LED) display, and any combinations thereof. Display device 176 may include, but is not limited to, a smartphone, tablet, laptop, monitor, tablet, and the like. Display device 176 may include a separate device that includes a transparent screen configured to display computer-generated images and/or information. In one or more embodiments, display device 176 may be configured to visually present data through a user interface or a graphical user interface (GUI) to at least an entity, such as first entity 128, second entity 144, and/or third entity 164, wherein the at least an entity may interact with the data through the user interface or GUI, as described below. In one or more embodiments, an entity may view GUI through display device 176. In one or more embodiments, display device 176 may be located on remote device, as described below. Additional details will be provided in this disclosure through nonlimiting examples.

With continued reference to FIG. 1, display device 176 may include a remote device. For the purposes of this disclosure, a “remote device” is a computer device separate and distinct from apparatus 100. For example, and without limitation, remote device may include a smartphone, a tablet, a laptop, a desktop computer, or the like. In one or more embodiments, remote device may be communicatively connected to apparatus 100 such as, for example, through network communication, through Bluetooth communication, and/or the like. In one or more embodiments, processor 104 may receive input data structure 132 and/or query response 160a-n to initiate one or more of subsequent steps through remote device. In one or more embodiments, one or more inputs 132 or responses 160a-n from one or more entities may be submitted through a user interface, such as a GUI, displayed through remote device, as described below.

With continued reference to FIG. 1, apparatus 100 comprises a user interface, wherein displaying recommended course of action 172 includes displaying the recommended course of action 172 through the user interface. In one or more embodiments, displaying recommended course of action 172 may include generating recommended course of action 172 and displaying, using display device 176, the recommended course of action 172 through user interface. For the purposes of this disclosure, a “user interface” is a means by which a user or entity and a computer system interact, for example, using input devices and software. User interface may include a graphical user interface (GUI), command line interface (CLI), menu-driven user interface, touch user interface, voice user interface (VUI), form-based user interface, any combination thereof, or the like. In one or more embodiments, a user may interact with user interface using computing device distinct from and communicatively connected to processor 104, such as a smartphone, tablet, or the like operated by the user. User interface may include one or more graphical locator and/or cursor facilities allowing user to interact with graphical models and/or combinations thereof, for instance using a touchscreen, touchpad, mouse, keyboard, and/or other manual data entry device. For the purposes of this disclosure, a “graphical user interface (GUI)” is a type of user interface that allows end users to interact with electronic devices through visual representations. In one or more embodiments, GUI may include icons, menus, other visual indicators or representations (graphics), audio indicators such as primary notation, display information, and related user controls. Menu may contain a list of choices and may allow users to select one from them. A menu bar may be displayed horizontally across the screen as a pull-down menu. Menu may include a context menu that appears only when user performs a specific action. Files, programs, web pages, and the like may be represented using a small picture within GUI. In one or more embodiments, GUI may include a graphical visualization of a user profile and/or the like. In one or more embodiments, processor 104 may be configured to modify and/or update GUI as a function of at least an input or the like by populating a user interface data structure and visually presenting data through modification of the GUI.

With continued reference to FIG. 1, in one or more embodiments, user interface and/or GUI may contain one or more interactive elements. For the purposes of this disclosure, an “interactive element” is an element within user interface and/or GUI that allows for communication with processor 104 by one or more users or entities. For example, and without limitation, interactive elements may include a plurality of tabs wherein selection of a particular tab, such as for example, by using a fingertip, may indicate to a system to perform a particular function and display the result through GUI. In one or more embodiments, interactive element may include tabs within GUI, wherein the selection of a particular tab may result in a particular function. In one or more embodiments, interactive elements may include words, phrases, illustrations, and the like to indicate a particular process that one or more users or entities would like system to perform. A person of ordinary skill in the art, upon reviewing the entirety of this disclosure, will be aware of various ways in which user interfaces, GUIs, and/or elements thereof may be implemented and/or used as described in this disclosure.

With continued reference to FIG. 1, in one or more embodiments, display device 176 and/or remote device may be configured to display at least an event handler graphic corresponding to at least an event handler. For the purposes of this disclosure, an “event handler graphic” is a graphical element with which user may interact using display device 176 and/or remote device to enter data, for instance and without limitation, for input data structure 132, query response 160a-n, or the like, as described above. Event handler graphic may include, without limitation, a button, a link, a checkbox, a text entry box and/or window, a drop-down list, a slider, or any other event handler graphic deemed suitable by a person of ordinary skill in the art upon reviewing the entirety of this disclosure. For the purposes of this disclosure, an “event handler” is a module, data structure, function, and/or routine that performs an action on display device 176 and/or remote device in response to one or more user inputs. For instance, and without limitation, event handler may record data corresponding to user selections of previously populated fields such as drop-down lists and/or text auto-complete and/or default entries, data corresponding to user selections of checkboxes, radio buttons, or the like, potentially along with automatically entered data triggered by such selections, user entry of textual data using a keyboard, touchscreen, speech-to-text program, or the like. Event handler may generate prompts for further information, may compare data to validation rules such as requirements that the data in question be entered within certain numerical ranges, and/or may modify data and/or generate warnings to user in response to such requirements. Event handler may convert data into expected and/or desired formats, for instance such as date formats, currency entry formats, name formats, or the like. Event handler may transmit data from a remote device to computing device.

With continued reference to FIG. 1, in one or more embodiments, event handler may include a cross-session state variable. For the purposes of this disclosure, a “cross-session state variable” is a variable recording data entered on remote device during a previous session. Such data may include, for instance, previously entered text, previous selections of one or more elements as described above, or the like. For instance, cross-session state variable data may represent a search that user entered in a past session. Cross-session state variable may be saved using any suitable combination of client-side data storage on remote device and server-side data storage on computing device; for instance, data may be saved wholly or in part as a “cookie” which may include data or an identification of remote device to prompt provision of cross-session state variable by the computing device, which may store the data on the computing device. Alternatively, or additionally, computing device may use login credentials, device identifier, and/or device fingerprint data to retrieve cross-session state variable, which the computing device may transmit to remote device. Cross-session state variable may include at least a prior session datum. A prior session datum may include any element of data that may be stored in cross-session state variable. Event handler graphic may be further configured to display at least a prior session datum, for instance and without limitation, by auto-populating user query data from previous sessions.

With continued reference to FIG. 1, in one or more embodiments, processor 104 and/or computing device may configure display device 176 and/or remote device to generate a graphical view. For the purposes of this disclosure, a “graphical view” is a data structure that results in display of one or more graphical elements on a screen. Graphical view may include at least a display element. For the purposes of this disclosure, a “display element” is an image that a program and/or data structure may cause to be displayed. Display elements may include, without limitation, windows, pop-up boxes, web browser pages, display layers, and/or any other display element deemed relevant by a person of ordinary skill in the art upon reviewing the entirety of this disclosure. Graphical view may include at least a selectable event graphic corresponding to one or more selectable event handlers. For the purposes of this disclosure, a “selectable event graphic” is a graphical element that, upon selection using a cursor or other locator as manipulated using a locator device such as a mouse, touchscreen, track pad, joystick, or the like, will trigger an action to be performed. As a nonlimiting example, a selectable event graphic may include a redirection link, defined as a hyperlink, button, image, portion of an image, and/or other graphic containing or referring to a uniform resource locator (URL) and/or other resource locator to another graphical view including without limitation buttons, and/or to a process that performs navigation to such URL and/or other resource locator upon selection of selectable event graphic. Redirection may be performed using any event handler, including without limitation event handlers detecting the click of a mouse or other locator, access of redirection link using a touchscreen, the selection of any key, mouseover events, or the like.

With continued reference to FIG. 1, it should be noted that apparatus 100 and related methods described herein are not limited to healthcare-related applications only. For example, and without limitation, apparatus 100 may be used to orchestrate applications such as recruiting, training, and managing contractors, coordinating complex manufacturing processes that requires multiples sources and areas of expertise, and organizing applicant pools in college or medical school admissions, among others. A person of ordinary skill in the art, upon reviewing the entirety of this disclosure, will recognize one or more embodiments described herein (although principally focused on healthcare-related applications) and their underlaying principles may be readily transferrable to a broader context of data management that is not currently disclosed.

With continued reference to FIG. 1, it is worth noting that apparatus 100 and related methods described herein are not limited to automating pre-procedural coordination and pre-admission testing workflows only. As a nonlimiting example, apparatus 100 may be used for organizing training sessions for nurses and/or other medical professionals. As another nonlimiting example, apparatus 100 may be used for streamlining quality assurance procedures and accelerating compliance checks. A person of ordinary skill in the art, upon reviewing the entirety of this disclosure, will recognize one or more embodiments described herein (although principally focused on management of pre-admission workflows) and their underlaying principles may be readily transferrable to a broader context of data management that is not currently disclosed.

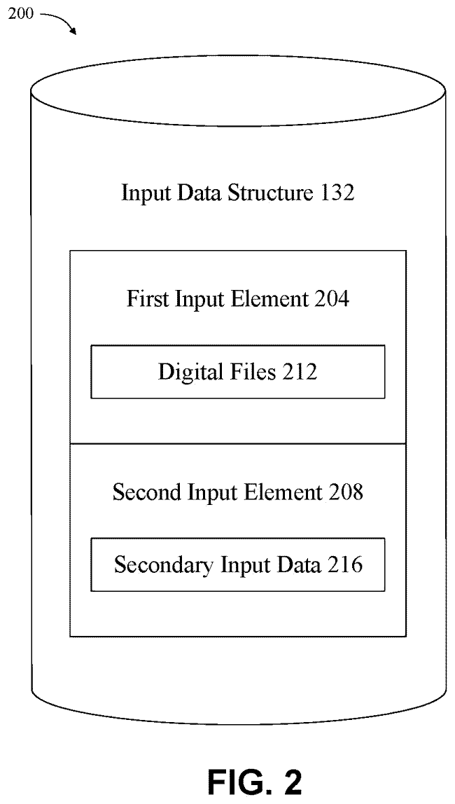

Referring now to FIG. 2, a block diagram of an exemplary embodiment 200 of input data structure 132 is illustrated. In one or more embodiments, input data structure 132 may include first input element 204 and second input element 208. In some cases, first input element 204 may include one or more digital files 212. In some cases, second input element 208 may include secondary input data 216, such as audio and/or textual secondary input data 216. Organization within and implementation of input data structure 132 may be consistent with details described above in this disclosure.

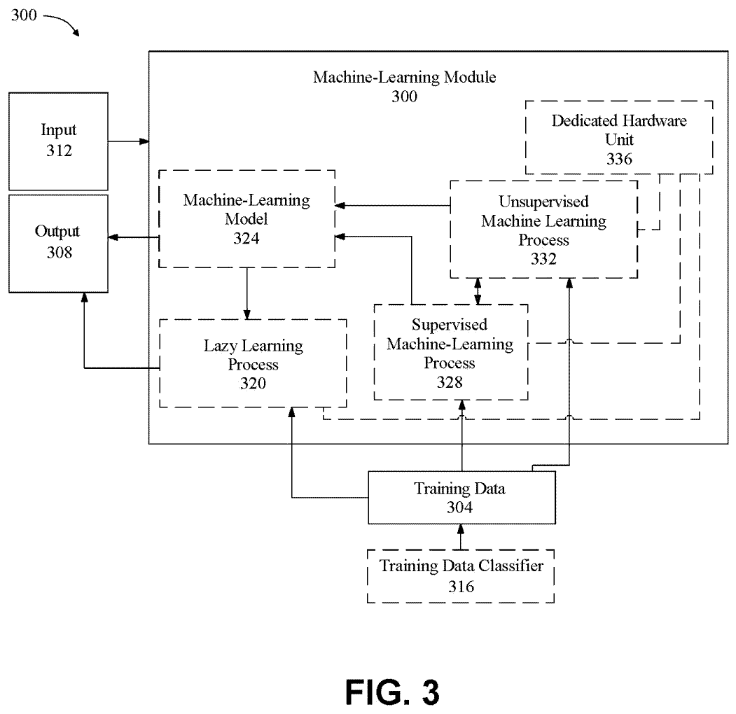

Referring now to FIG. 3, an exemplary embodiment of a machine learning module 300 that may perform one or more machine learning processes as described above is illustrated. Machine learning module may perform determinations, classification, and/or analysis steps, methods, processes, or the like as described in this disclosure using machine learning processes. For the purposes of this disclosure, a “machine learning process” is an automated process that uses training data 304 to generate an algorithm instantiated in hardware or software logic, data structures, and/or functions that will be performed by a computing device/module to produce outputs 308 given data provided as inputs 312; this is in contrast to a non-machine learning software program where the commands to be executed are pre-determined by user and written in a programming language.

With continued reference to FIG. 3, “training data”, for the purposes of this disclosure, are data containing correlations that a machine learning process may use to model relationships between two or more categories of data elements. For instance, and without limitation, training data 304 may include a plurality of data entries, also known as “training examples”, each entry representing a set of data elements that were recorded, received, and/or generated together. Data elements may be correlated by shared existence in a given data entry, by proximity in a given data entry, or the like. Multiple data entries in training data 304 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 304 according to various correlations; correlations may indicate causative and/or predictive links between categories of data elements, which may be modeled as relationships such as mathematical relationships by machine learning processes as described in further detail below. Training data 304 may be formatted and/or organized by categories of data elements, for instance by associating data elements with one or more descriptors corresponding to categories of data elements. As a nonlimiting example, training data 304 may include data entered in standardized forms by persons or processes, such that entry of a given data element within a given field in a given form may be mapped to one or more descriptors of categories. Elements in training data 304 may be linked to descriptors of categories by tags, tokens, or other data elements. For instance, and without limitation, training data 304 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

With continued reference to FIG. 3, alternatively or additionally, training data 304 may include one or more elements that are uncategorized; that is, training data 304 may not be formatted or contain descriptors for some elements of data. Machine learning algorithms and/or other processes may sort training data 304 according to one or more categorizations using, for instance, natural language processing algorithms, tokenization, detection of correlated values in raw data, and the like; categories may be generated using correlation and/or other processing algorithms. As a nonlimiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine learning algorithms, and/or automated association of data in the data entry with descriptors or into a given format. The ability to categorize data entries automatedly may enable the same training data 304 to be made applicable for two or more distinct machine learning algorithms as described in further detail below. Training data 304 used by machine learning module 300 may correlate any input data as described in this disclosure to any output data as described in this disclosure. As a nonlimiting illustrative example, inputs may include one or more training examples such medical literature and/or EHRs, whereas outputs may include one or more content retrieval parameters 124a-n.

With continued reference to FIG. 3, training data 304 may be filtered, sorted, and/or selected using one or more supervised and/or unsupervised machine learning processes and/or models as described in further detail below; such processes and/or models may include without limitation a training data classifier 316. For the purposes of this disclosure, a “classifier” is a machine learning model, such as a data structure representing and/or using a mathematical model, neural net, or a program generated by a machine learning algorithm, known as a “classification algorithm”, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine learning module 300 may generate a classifier using a classification algorithm. For the purposes of this disclosure, a “classification algorithm” is a process wherein a computing device and/or any module and/or component operating therein derives a classifier from training data 304. Classification may be performed using, without limitation, linear classifiers such as without limitation logistic regression and/or naive Bayes classifiers, nearest neighbor classifiers such as k-nearest neighbors classifiers, support vector machines, least squares support vector machines, Fisher's linear discriminant, quadratic classifiers, decision trees, boosted trees, random forest classifiers, learning vector quantization, and/or neural network-based classifiers. In one or more embodiments, training data classifier 316 may classify elements of training data to a plurality of cohorts as a function of certain anatomic and/or demographic traits.

With continued reference to FIG. 3, machine learning module 300 may be configured to generate a classifier using a naive Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naive Bayes classification algorithm may be based on Bayes Theorem expressed as P(A/B)=P(B/A)× P(A)+P(B), where P(A/B) is the probability of hypothesis A given data B, also known as posterior probability; P(B/A) is the probability of data B given that the hypothesis A was true; P(A) is the probability of hypothesis A being true regardless of data, also known as prior probability of A; and P(B) is the probability of the data regardless of the hypothesis. A naive Bayes algorithm may be generated by first transforming training data into a frequency table. Machine learning module 300 may then calculate a likelihood table by calculating probabilities of different data entries and classification labels. Machine learning module 300 may utilize a naive Bayes equation to calculate a posterior probability for each class. A class containing the highest posterior probability is the outcome of prediction. Naïve Bayes classification algorithm may include a gaussian model that follows a normal distribution. Naive Bayes classification algorithm may include a multinomial model that is used for discrete counts. Naive Bayes classification algorithm may include a Bernoulli model that may be utilized when vectors are binary.

With continued reference to FIG. 3, machine learning module 300 may be configured to generate a classifier using a k-nearest neighbors (KNN) algorithm. For the purposes of this disclosure, a “k-nearest neighbors algorithm” is or at least includes a classification method that utilizes feature similarity to analyze how closely out-of-sample features resemble training data 304 and to classify input data to one or more clusters and/or categories of features as represented in training data 304; this may be performed by representing both training data 304 and input data in vector forms and using one or more measures of vector similarity to identify classifications within training data 304 and determine a classification of input data. K-nearest neighbors algorithm may include specifying a k-value, or a number directing the classifier to select the k most similar entries of training data 304 to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a nonlimiting example, an initial heuristic may include a ranking of associations between inputs 312 and elements of training data 304. Heuristic may include selecting some number of highest-ranking associations and/or training data elements.

With continued reference to FIG. 3, generating k-nearest neighbors algorithm may generate a first vector output containing a data entry cluster, generating a second vector output containing input data, and calculate the distance between the first vector output and the second vector output using any suitable norm such as cosine similarity, Euclidean distance measurement, or the like. Each vector output may be represented, without limitation, as an n-tuple of values, where n is at least 2. Each value of n-tuple of values may represent a measurement or other quantitative value associated with a given category of data or attribute, examples of which are provided in further detail below. A vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent when their directions and/or relative quantities of values are the same; thus, as a nonlimiting example, a vector represented as [5, 10, 15] may be treated as equivalent, for the purposes of this disclosure, as a vector represented as [1, 2, 3]. Vectors may be more similar where their directions are more similar, and more different where their directions are more divergent. However, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized”, or divided by a “length” attribute, such as a length attribute l as derived using a Pythagorean norm:

$$l = \sqrt{{Σ}_{i = 0}^{n} {a}_{i}^{2}} ,$$

where aᵢ is attribute number of vector i. Scaling and/or normalization may function to make vector comparison independent of absolute quantities of attributes, while preserving any dependency on similarity of attributes. This may, for instance, be advantageous where cases represented in training data 304 are represented by different quantities of samples, which may result in proportionally equivalent vectors with divergent values.

With continued reference to FIG. 3, training examples for use as training data may be selected from a population of potential examples according to cohorts relevant to an analytical problem to be solved, a classification task, or the like. Alternatively or additionally, training data 304 may be selected to span a set of likely circumstances or inputs for a machine learning model and/or process to encounter when deployed. For instance, and without limitation, for each category of input data to a machine learning model and/or process that may exist in a range of values in a population of phenomena such as images, user data, process data, physical data, or the like, a computing device, processor 104, and/or machine learning module 300 may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to user, so that a process can detect, automatically or via user input, one or more values that are not included in the set of training examples. Computing device, processor 104, and/or machine learning module 300 may automatically generate a missing training example. This may be done by receiving and/or retrieving a missing input and/or output value and correlating the missing input and/or output value with a corresponding output and/or input value collocated in a data record with the retrieved value, provided by user, another device, or the like.

With continued reference to FIG. 3, computing device, processor 104, and/or machine learning module 300 may be configured to preprocess training data 304. For the purposes of this disclosure, “preprocessing” training data is a process that transforms training data from a raw form to a format that can be used for training a machine learning model. Preprocessing may include sanitizing, feature selection, feature scaling, data augmentation and the like.

With continued reference to FIG. 3, computing device, processor 104, and/or machine learning module 300 may be configured to sanitize training data. For the purposes of this disclosure, “sanitizing” training data is a process whereby training examples that interfere with convergence of a machine learning model and/or process are removed to yield a useful result. For instance, and without limitation, a training example may include an input and/or output value that is an outlier from typically encountered values, such that a machine learning algorithm using the training example will be skewed to an unlikely range of input 312 and/or output 308; a value that is more than a threshold number of standard deviations away from an average, mean, or expected value, for instance, may be eliminated. Alternatively or additionally, one or more training examples may be identified as having poor-quality data, where “poor-quality” means having a signal-to-noise ratio below a threshold value. In one or more embodiments, sanitizing training data may include steps such as removing duplicative or otherwise redundant data, interpolating missing data, correcting data errors, standardizing data, identifying outliers, and/or the like. In one or more embodiments, sanitizing training data may include algorithms that identify duplicate entries or spell-check algorithms.

With continued reference to FIG. 3, in one or more embodiments, images used to train an image classifier or other machine learning model and/or process that takes images as inputs 312 or generates images as outputs 308 may be rejected if image quality is below a threshold value. For instance, and without limitation, computing device, processor 104, and/or machine learning module 300 may perform blur detection. Elimination of one or more blurs may be performed, as a nonlimiting example, by taking Fourier transform or a Fast Fourier Transform (FFT) of image and analyzing a distribution of low and high frequencies in the resulting frequency-domain depiction of the image. Numbers of high-frequency values below a threshold level may indicate blurriness. As a further nonlimiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of image, based on the hypothesis that rapid changes indicate sharp edges in the image, and thus are indicative of a lower degree of blurriness. Blur detection may be performed using a wavelet-based operator, which uses coefficients of a discrete wavelet transform to describe the frequency and spatial content of images. Blur detection may be performed using statistics-based operators that take advantage of several image statistics as texture descriptors in order to compute a focus level. Blur detection may be performed by using discrete cosine transform (DCT) coefficients in order to compute a focus level of an image from its frequency content.

With continued reference to FIG. 3, computing device, processor 104, and/or machine learning module 300 may be configured to precondition one or more training examples. For instance, and without limitation, where a machine learning model and/or process has one or more inputs 312 and/or outputs 308 requiring, transmitting, or receiving a certain number of bits, samples, or other units of data, one or more elements of training examples to be used as or compared to inputs 312 and/or outputs 308 may be modified to have such a number of units of data. In one or more embodiments, computing device, processor 104, and/or machine learning module 300 may convert a smaller number of units, such as in a low pixel count image, into a desired number of units by upsampling and interpolating. As a nonlimiting example, a low pixel count image may have 100 pixels, whereas a desired number of pixels may be 128. Processor 104 may interpolate the low pixel count image to convert 100 pixels into 128 pixels. It should also be noted that one of ordinary skill in the art, upon reading the entirety of this disclosure, would recognize the various methods to interpolate a smaller number of data units such as samples, pixels, bits, or the like to a desired number of such units. In one or more embodiments, a set of interpolation rules may be trained by sets of highly detailed inputs 312 and/or outputs 308 and corresponding inputs 312 and/or outputs 308 downsampled to smaller numbers of units, and a neural network or another machine learning model that is trained to predict interpolated pixel values using the training data 304. As a nonlimiting example, a sample input 312 and/or output 308, such as a sample picture, with sample-expanded data units (e.g., pixels added between the original pixels) may be input to a neural network or machine learning model and output a pseudo replica sample picture with dummy values assigned to pixels between the original pixels based on a set of interpolation rules. As a nonlimiting example, in the context of an image classifier, a machine learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, computing device, processor 104, and/or machine learning module 300 may utilize sample expander methods, a low-pass filter, or both. For the purposes of this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor 104, and/or machine learning module 300 may use averaging, such as luma or chroma averaging in images, to fill in data units in between original data units.

With continued reference to FIG. 3, in one or more embodiments, computing device, processor 104, and/or machine learning module 300 may downsample elements of a training example to a desired lower number of data elements. As a nonlimiting example, a high pixel count image may contain 256 pixels, however a desired number of pixels may be 128. Processor 104 may downsample the high pixel count image to convert 256 pixels into 128 pixels. In one or more embodiments, processor 104 may be configured to perform downsampling on data. Downsampling, also known as decimation, may include removing every N^{th} entry in a sequence of samples, all but every N^{th} entry, or the like, which is a process known as “compression” and may be performed, for instance by an N-sample compressor implemented using hardware or software. Anti-aliasing and/or anti-imaging filters, and/or low-pass filters, may be used to eliminate side effects of compression.

With continued reference to FIG. 3, feature selection may include narrowing and/or filtering training data 304 to exclude features and/or elements, or training data including such elements that are not relevant to a purpose for which a trained machine learning model and/or algorithm is being trained, and/or collection of features, elements, or training data including such elements based on relevance to or utility for an intended task or purpose for which a machine learning model and/or algorithm is being trained. Feature selection may be implemented, without limitation, using any process described in this disclosure, including without limitation using training data classifiers, exclusion of outliers, or the like.

With continued reference to FIG. 3, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, wherein a difference between each value, X, and a minimum value, Xₘᵢₙ, in a set or subset of values is divided by a range of values, Xₘₐₓ-Xₘᵢₙ, in the set or subset:

$${X}_{new} = \frac{X - {X}_{min}}{{X}_{max} - {X}_{min}} .$$

Feature scaling may include mean normalization, wherein a difference between each value, X, and a mean value of a set and/or subset of values, Xₘₑₐₙ, is divided by a range of values, Xₘₐₓ-Xₘᵢₙ, in the set or subset:

$${X}_{n e w} = \frac{X - {X}_{m e a n}}{{X}_{max} - {X}_{min}} .$$

Feature scaling may include standardization, wherein a difference between X and Xₘₑₐₙ is divided by a standard deviation, σ, of a set or subset of values:

$${X}_{n e w} = \frac{X - {X}_{m e a n}}{σ} .$$

Feature scaling may be performed using a median value of a set or subset, X_{median}, and/or interquartile range (IQR), which represents the difference between the 25th percentile value and the 50th percentile value (or closest values thereto by a rounding protocol), such as:

$${X}_{n e w} = \frac{X - {X}_{m e d i a n}}{IQR} .$$

A Person of ordinary skill in the art, upon reviewing the entirety of this disclosure, will be aware of various alternative or additional approaches that may be used for feature scaling.

With continued reference to FIG. 3, computing device, processor 104, and/or machine learning module 300 may be configured to perform one or more processes of data augmentation. For the purposes of this disclosure, “data augmentation” is a process that adds data to a training data 304 using elements and/or entries already in the dataset. Data augmentation may be accomplished, without limitation, using interpolation, generation of modified copies of existing entries and/or examples, and/or one or more generative artificial intelligence (AI) processes, for instance using deep neural networks and/or generative adversarial networks. Generative processes may be referred to alternatively in this context as “data synthesis” and as creating “synthetic data”. Augmentation may include performing one or more transformations on data, such as geometric, color space, affine, brightness, cropping, and/or contrast transformations of images.

With continued reference to FIG. 3, machine learning module 300 may be configured to perform a lazy learning process and/or protocol 320. For the purposes of this disclosure, a “lazy learning” process and/or protocol is a process whereby machine learning is conducted upon receipt of input 312 to be converted to output 308 by combining the input 312 and training data 304 to derive the algorithm to be used to produce the output 308 on demand. A lazy learning process may alternatively be referred to as a “lazy loading” or “call-when-needed” process and/or protocol. For instance, an initial set of simulations may be performed to cover an initial heuristic and/or “first guess” at an output 308 and/or relationship. As a nonlimiting example, an initial heuristic may include a ranking of associations between inputs 312 and elements of training data 304. Heuristic may include selecting some number of highest-ranking associations and/or training data 304 elements. Lazy learning may implement any suitable lazy learning algorithm, including without limitation a k-nearest neighbors algorithm, a lazy naive Bayes algorithm, or the like. A person of ordinary skill in the art, upon reviewing the entirety of this disclosure, will be aware of various lazy learning algorithms that may be applied to generate outputs as described in this disclosure, including without limitation lazy learning applications of machine learning algorithms as described in further detail below.

With continued reference to FIG. 3, alternatively or additionally, machine learning processes as described in this disclosure may be used to generate machine learning models 324. A “machine learning model”, for the purposes of this disclosure, is a data structure representing and/or instantiating a mathematical and/or algorithmic representation of a relationship between inputs 312 and outputs 308, generated using any machine learning process including without limitation any process described above, and stored in memory. An input 312 is submitted to a machine learning model 324 once created, which generates an output 308 based on the relationship that was derived. For instance, and without limitation, a linear regression model, generated using a linear regression algorithm, may compute a linear combination of input data using coefficients derived during machine learning processes to calculate an output datum. As a further nonlimiting example, a machine learning model 324 may be generated by creating an artificial neural network, such as a convolutional neural network comprising an input layer of nodes, one or more intermediate layers, and an output layer of nodes. Connections between nodes may be created by “training” the network, in which elements from a training data 304 are applied to the input nodes, and a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning, as described in detail below.

With continued reference to FIG. 3, machine learning module 300 may perform at least a supervised machine learning process 328. For the purposes of this disclosure, a “supervised” machine learning process is a process with algorithms that receive training data 304 relating one or more inputs 312 to one or more outputs 308, and seek to generate one or more data structures representing and/or instantiating one or more mathematical relations relating input 312 to output 308, where each of the one or more mathematical relations is optimal according to some criterion specified to the algorithm using some scoring function. For instance, a supervised learning algorithm may include inputs 312 described above as inputs, and outputs 308 described above as outputs, and a scoring function representing a desired form of relationship to be detected between inputs 312 and outputs 308. Scoring function may, for instance, seek to maximize the probability that a given input 312 and/or combination thereof is associated with a given output 308 to minimize the probability that a given input 312 is not associated with a given output 308. Scoring function may be expressed as a risk function representing an “expected loss” of an algorithm relating inputs 312 to outputs 308, where loss is computed as an error function representing a degree to which a prediction generated by the relation is incorrect when compared to a given input-output pair provided in training data 304. Supervised machine learning processes may include classification algorithms as defined above. A person of ordinary skill in the art, upon reviewing the entirety of this disclosure, will be aware of various possible variations of at least a supervised machine learning process 328 that may be used to determine a relation between inputs and outputs.

With continued reference to FIG. 3, training a supervised machine learning process may include, without limitation, iteratively updating coefficients, biases, and weights based on an error function, expected loss, and/or risk function. For instance, an output 308 generated by a supervised machine learning model 328 using an input example in a training example may be compared to an output example from the training example; an error function may be generated based on the comparison, which may include any error function suitable for use with any machine learning algorithm described in this disclosure, including a square of a difference between one or more sets of compared values or the like. Such an error function may be used in turn to update one or more weights, biases, coefficients, or other parameters of a machine learning model through any suitable process including without limitation gradient descent processes, least-squares processes, and/or other processes described in this disclosure. This may be done iteratively and/or recursively to gradually tune such weights, biases, coefficients, or other parameters. Updates may be performed in neural networks using one or more back-propagation algorithms. Iterative and/or recursive updates to weights, biases, coefficients, or other parameters as described above may be performed until currently available training data 304 are exhausted and/or until a convergence test is passed. For the purposes of this disclosure, a “convergence test” is a test for a condition selected to indicate that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

With continued reference to FIG. 3, a computing device, processor 104, and/or machine learning module 300 may be configured to perform method, method step, sequence of method steps, and/or algorithm described in reference to this figure, in any order and with any degree of repetition. For instance, computing device, processor 104, and/or machine learning module 300 may be configured to perform a single step, sequence, and/or algorithm repeatedly until a desired or commanded outcome is achieved; repetition of a step or a sequence of steps may be performed iteratively and/or recursively using outputs 308 of previous repetitions as inputs 312 to subsequent repetitions, aggregating inputs 312 and/or outputs 308 of repetitions to produce an aggregate result, reduction or decrement of one or more variables such as global variables, and/or division of a larger processing task into a set of iteratively addressed smaller processing tasks. A computing device, processor 104, apparatus 100, or machine learning module 300 may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. A person of ordinary skill in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 3, machine learning process may include at least an unsupervised machine learning process 332. For the purposes of this disclosure, an unsupervised machine learning process is a process that derives inferences in datasets without regard to labels. As a result, an unsupervised machine learning process 332 may be free to discover any structure, relationship, and/or correlation provided in the data. Unsupervised processes 332 may not require a response variable, may be used to find interesting patterns and/or inferences between variables, to determine a degree of correlation between two or more variables, or the like.

With continued reference to FIG. 3, machine learning module 300 may be designed and configured to create machine learning model 324 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include an elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to a person of ordinary skill in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought. Similar methods to those described above may be applied to minimize error functions, as will be apparent to a person of ordinary skill in the art upon reviewing the entirety of this disclosure.

With continued reference to FIG. 3, machine learning algorithms may include, without limitation, linear discriminant analysis. Machine learning algorithm may include quadratic discriminant analysis. Machine learning algorithms may include kernel ridge regression. Machine learning algorithms may include support vector machines, including without limitation support vector classification-based regression processes. Machine learning algorithms may include stochastic gradient descent algorithms, including classification and regression algorithms based on stochastic gradient descent. Machine learning algorithms may include nearest neighbors algorithms. Machine learning algorithms may include various forms of latent space regularization such as variational regularization. Machine learning algorithms may include Gaussian processes such as Gaussian Process Regression. Machine learning algorithms may include cross-decomposition algorithms, including partial least squares and/or canonical correlation analysis. Machine learning algorithms may include naïve Bayes methods. Machine learning algorithms may include algorithms based on decision trees, such as decision tree classification or regression algorithms. Machine learning algorithms may include ensemble methods such as bagging meta-estimator, forest of randomized trees, AdaBoost, gradient tree boosting, and/or voting classifier methods. Machine learning algorithms may include neural net algorithms, including convolutional neural net processes.

With continued reference to FIG. 3, a machine learning model and/or process may be deployed or instantiated by incorporation into a program, apparatus, system, and/or module. For instance, and without limitation, a machine learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit, to represent a number according to any suitable encoding system including twos complement or the like, or may be stored in any volatile and/or non-volatile memory. Similarly, mathematical operations and input 312 and/or output 308 of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine learning process and/or model, including without limitation any combination of production and/or configuration of non-reconfigurable hardware elements, circuits, and/or modules such as without limitation application-specific integrated circuits (ASICs), production and/or configuration of reconfigurable hardware elements, circuits, and/or modules such as without limitation field programmable gate arrays (FPGAs), production and/or configuration of non-reconfigurable and/or non-rewritable memory elements, circuits, and/or modules such as without limitation non-rewritable read-only memory (ROM), other memory technology described in this disclosure, and/or production and/or configuration of any computing device and/or component thereof as described in this disclosure. Such deployed and/or instantiated machine learning model and/or algorithm may receive inputs 312 from any other process, module, and/or component described in this disclosure, and produce outputs 308 to any other process, module, and/or component described in this disclosure.

With continued reference to FIG. 3, any process of training, retraining, deployment, and/or instantiation of any machine learning model and/or algorithm may be performed and/or repeated after an initial deployment and/or instantiation to correct, refine, and/or improve the machine learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs 308 of machine learning models and/or algorithms, and/or errors and/or error functions thereof, to any thresholds, convergence tests, or the like, and/or may compare outputs 308 of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

With continued reference to FIG. 3, retraining and/or additional training may be performed using any process for training described above, using any currently or previously deployed version of a machine learning model and/or algorithm as a starting point. Training data for retraining may be collected, preconditioned, sorted, classified, sanitized, or otherwise processed according to any process described in this disclosure. Training data 304 may include, without limitation, training examples including inputs 312 and correlated outputs 308 used, received, and/or generated from any version of any system, module, machine learning model or algorithm, apparatus, and/or method described in this disclosure. Such examples may be modified and/or labeled according to user feedback or other processes to indicate desired results, and/or may have actual or measured results from a process being modeled and/or predicted by system, module, machine learning model or algorithm, apparatus, and/or method as “desired” results to be compared to outputs 308 for training processes as described above. Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

With continued reference to FIG. 3, one or more processes or algorithms described above may be performed by at least a dedicated hardware unit 336. For the purposes of this disclosure, a “dedicated hardware unit” is a hardware component, circuit, or the like, aside from a principal control circuit and/or processor 104 performing method steps as described in this disclosure, that is specifically designated or selected to perform one or more specific tasks and/or processes described in reference to this figure, such as without limitation preprocessing and/or sanitization of training data and/or training a machine learning algorithm and/or model. Dedicated hardware unit 336 may include, without limitation, a hardware unit that can perform iterative or massed calculations, such as matrix-based calculations to update or tune parameters, weights, coefficients, and/or biases of machine learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously, in parallel, and/or the like. Such dedicated hardware units 336 may include, without limitation, graphical processing units (GPUs), dedicated signal processing modules, field programmable gate arrays (FPGA), other reconfigurable hardware that has been configured to instantiate parallel processing units for one or more specific tasks, or the like. Computing device, processor 104, apparatus 100, or machine learning module 300 may be configured to instruct one or more dedicated hardware units 336 to perform one or more operations described herein, such as evaluation of model and/or algorithm outputs, one-time or iterative updates to parameters, coefficients, weights, and/or biases, vector and/or matrix operations, and/or any other operations described in this disclosure.

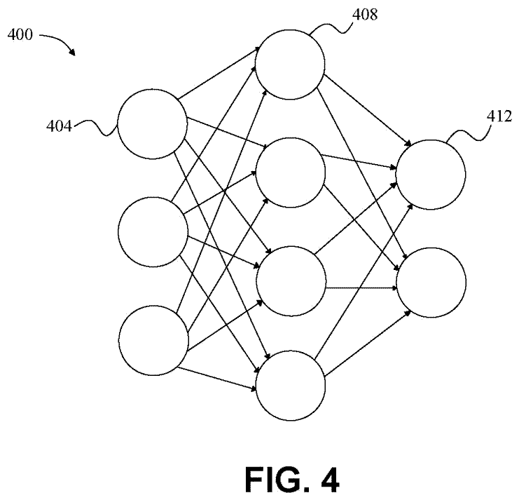

Referring now to FIG. 4, an exemplary embodiment of neural network 400 is illustrated. For the purposes of this disclosure, a neural network or artificial neural network is a network of “nodes” or data structures having one or more inputs, one or more outputs, and a function determining outputs based on inputs. Such nodes may be organized in a network, such as without limitation a convolutional neural network, including an input layer of nodes 404, at least an intermediate layer of nodes 408, and an output layer of nodes 412. Connections between nodes may be created via the process of “training” neural network 400, in which elements from a training dataset are applied to the input nodes, and a suitable training algorithm (such as Levenberg-Marquardt, conjugate gradient, simulated annealing, or other algorithms) is then used to adjust the connections and weights between nodes in adjacent layers of the neural network 400 to produce the desired values at the output nodes. This process is sometimes referred to as deep learning. Connections may run solely from input nodes toward output nodes in a “feed-forward” network or may feed outputs of one layer back to inputs of the same or a different layer in a “recurrent network”. As a further nonlimiting example, neural network 400 may include a convolutional neural network comprising an input layer of nodes 404, one or more intermediate layers of nodes 408, and an output layer of nodes 412. For the purposes of this disclosure, a “convolutional neural network” is a type of neural network 400 in which at least one hidden layer is a convolutional layer that convolves inputs to that layer with a subset of inputs known as a “kernel”, along with one or more additional layers such as pooling layers, fully connected layers, and the like.

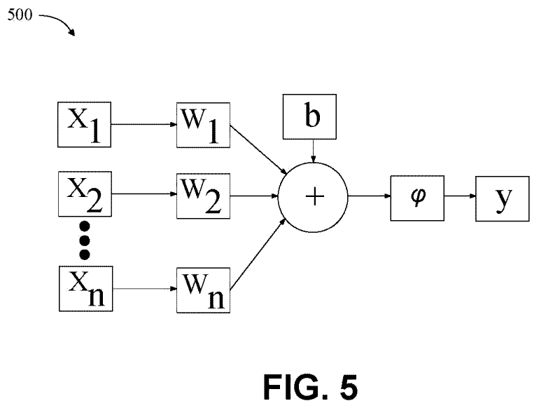

Referring now to FIG. 5, an exemplary embodiment of a node 500 of neural network 400 is illustrated. Node 500 may include, without limitation, a plurality of inputs, x¿, that may receive numerical values from inputs to neural network 400 containing the node 500 and/or from other nodes 500. Node 500 may perform one or more activation functions to produce its output given one or more inputs, such as without limitation computing a binary step function comparing an input to a threshold value and outputting either a logic 1 or logic 0 output or its equivalent, a linear activation function whereby an output is directly proportional to input, and/or a nonlinear activation function wherein the output is not proportional to the input. Nonlinear activation functions may include, without limitation, a sigmoid function of the form

$$f ( x ) = \frac{1}{1 - {e}^{- x}}$$

given input x, a tanh (hyperbolic tangent) function of the form

$$\frac{{e}^{x} - {e}^{- x}}{{e}^{x} + {e}^{- x}} ,$$

a tanh derivative function such as ƒ(x)=tanh²(x), a rectified linear unit function such as ƒ(x)=max(0, x), a “leaky” and/or “parametric” rectified linear unit function such as ƒ(x)=max(ax, x) for some value of a, an exponential linear units function such as

$$f ( x ) = \{ \begin{matrix}x for x \geq 0 \\ α ( {e}^{x} - 1 ) for x < 0\end{matrix}$$

for some value of α (this function may be replaced and/or weighted by its own derivative in some embodiments), a softmax function such as

$$f ( {x}_{i} ) = \frac{{e}^{x}}{{Σ}_{i} {x}_{i}}$$

where the inputs to an instant layer are xᵢ, a swish function such as ƒ(x)=x*sigmoid(x), a Gaussian error linear unit function such as ƒ(x)=a (1+tanh (\sqrt{2/π} (x+bx^{r}))) for some values of a, b, and r, and/or a scaled exponential linear unit function such as

$$f ( x ) = λ \{ \begin{matrix}α ( {e}^{x} - 1 ) for x < 0 \\ x for x \geq 0\end{matrix} .$$

Fundamentally, there is no limit to the nature of functions of inputs xᵢ, that may be used as activation functions. As a nonlimiting and illustrative example, node 500 may perform a weighted sum of inputs using weights, wᵢ, that are multiplied by respective inputs, xᵢ. Additionally or alternatively, a bias b may be added to the weighted sum of the inputs such that an offset is added to each unit in a neural network layer that is independent of the input to the layer. The weighted sum may then be input into a function, φ, which may generate one or more outputs, y. Weight, wᵢ, applied to an input, x¿, may indicate whether the input is “excitatory”, indicating that it has strong influence on the one or more outputs, y, for instance by the corresponding weight having a large numerical value, or “inhibitory”, indicating it has a weak influence on the one more outputs, y, for instance by the corresponding weight having a small numerical value. The values of weights, wᵢ, may be determined by training neural network 400 using training data, which may be performed using any suitable process as described above.

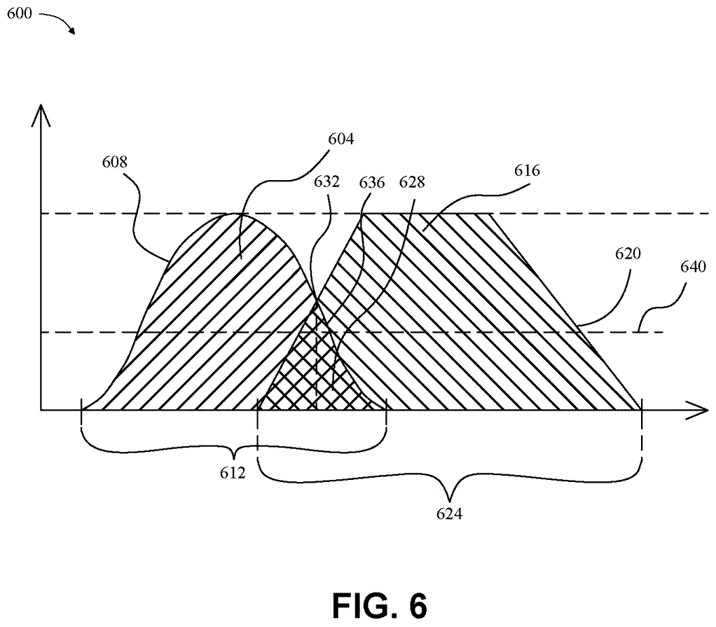

Referring now to FIG. 6, an exemplary embodiment of fuzzy set comparison 600 is illustrated. A first fuzzy set 604 may be represented, without limitation, according to a first membership function 608 representing a probability that an input falling on a first range of values 612 is a member of the first fuzzy set 604, where the first membership function 608 has values on a range of probabilities such as without limitation the interval [0,1], and an area beneath the first membership function 608 may represent a set of values within the first fuzzy set 604. Although first range of values 612 is illustrated for clarity in this exemplary depiction as a range on a single number line or axis, first range of values 612 may be defined on two or more dimensions, representing, for instance, a Cartesian product between a plurality of ranges, curves, axes, spaces, dimensions, or the like. First membership function 608 may include any suitable function mapping first range 612 to a probability interval, including without limitation a triangular function defined by two linear elements such as line segments or planes that intersect at or below the top of the probability interval. As a non-limiting example, triangular membership function may be defined as:

$$y ( x , a , b , c ) = \{ \begin{matrix}0 , for x > c and x < a \\ \frac{x - a}{b - a} , for a \leq x < b \\ \frac{c - x}{c - b} , if b < x \leq c\end{matrix}$$

a trapezoidal membership function may be defined as:

$$y ( x , a , b , c , d ) = max ( min ( \frac{x - a}{b - a} , 1 , \frac{d - x}{d - c} ) , 0 )$$

a sigmoidal function may be defined as:

$$y ( x , a , c ) = \frac{1}{1 - {e}^{- a ( x - c )}}$$

a Gaussian membership function may be defined as:

$$y ( x , c , σ ) = {e}^{- \frac{1}{2} {( \frac{x - c}{σ} )}^{2}}$$

and a bell membership function may be defined as:

$$y ( x , a , b , c , ) = {[ 1 + \vert \frac{x - c}{a} {\vert}^{2 b} ]}^{- 1}$$

A person of ordinary skill in the art, upon reviewing the entirety of this disclosure, will be aware of various alternative or additional membership functions that may be used consistently with this disclosure.

With continued reference to FIG. 6, in one or more embodiments, first fuzzy set 604 may represent any value or combination of values as described above, including output from one or more machine learning models. A second fuzzy set 616, which may represent any value which may be represented by first fuzzy set 604, may be defined by a second membership function 620 on a second range 624; second range 624 may be identical and/or overlap with first range 612 and/or may be combined with first range via Cartesian product or the like to generate a mapping permitting evaluation overlap of first fuzzy set 604 and second fuzzy set 616. Where first fuzzy set 604 and second fuzzy set 616 have a region 628 that overlaps, first membership function 608 and second membership function 620 may intersect at a point 632 representing a probability, as defined on probability interval, of a match between first fuzzy set 604 and second fuzzy set 616. Alternatively, or additionally, a single value of first and/or second fuzzy set may be located at a locus 636 on first range 612 and/or second range 624, where a probability of membership may be taken by evaluation of first membership function 608 and/or second membership function 620 at that range point. A probability at 628 and/or 632 may be compared to a threshold 640 to determine whether a positive match is indicated. Threshold 640 may, in a nonlimiting example, represent a degree of match between first fuzzy set 604 and second fuzzy set 616, and/or single values therein with each other or with either set, which is sufficient for purposes of the matching process; for instance, threshold 640 may indicate a sufficient degree of overlap between an output from one or more machine learning models. Alternatively or additionally, each threshold 640 may be tuned by a machine learning and/or statistical process, for instance and without limitation as described in further detail in this disclosure.

With continued reference to FIG. 6, in one or more embodiments, a degree of match between fuzzy sets may be used to classify plurality of training examples, medical images, one or more textual or visual features therein, and/or the like, as described above in this disclosure. As a nonlimiting example, if one or more medical features are associated with a fuzzy set that matches a fuzzy set of a cohort by having a degree of overlap exceeding a threshold, computing device may classify the one or more medical feature as belonging to that cohort. Where multiple fuzzy matches are performed, degrees of match for each respective fuzzy set may be computed and aggregated through, for instance, addition, averaging, or the like, to determine an overall degree of match.

With continued reference to FIG. 6, in one or more embodiments, one or more medical features may be compared to multiple fuzzy sets of multiple cohorts. As a nonlimiting example, one or more medical features may be represented by a fuzzy set that is compared to each of the multiple fuzzy sets of multiple cohorts, and a degree of overlap exceeding a threshold between the fuzzy set representing the medical features and any of the multiple fuzzy sets representing multiple cohorts may cause computing device to classify the medical features as belonging to that cohort. As a nonlimiting example, there may be two fuzzy sets representing two cohorts, cohort A and cohort B. Cohort A may have a cohort A fuzzy set, cohort B may have a cohort B fuzzy set, and medical features may have a medical feature fuzzy set. Computing device may compare medical feature fuzzy set with each of cohort A fuzzy set and cohort B fuzzy set, as described above, and classify medical features to either, both, or neither of cohort A fuzzy set and cohort B fuzzy set. Machine learning methods as described throughout this disclosure may, in a nonlimiting example, generate coefficients used in fuzzy set equations as described above, such as without limitation x, c, and σ of a Gaussian set as described above, as outputs of machine learning methods. Likewise, medical features may be used indirectly to determine a fuzzy set, as medical feature fuzzy set may be derived from outputs of one or more machine learning models that take medical features directly or indirectly as inputs.

With continued reference to FIG. 6, in one or more embodiments, fuzzy set comparison 600 may include a fuzzy inference model. For the purposes of this disclosure, a “fuzzy inference model” is a model that uses fuzzy logic to reach a decision and derive a meaningful outcome. As a nonlimiting example, a fuzzy inference system may be associated with degrees of a medical condition, such as “Normal”, “Grade 1”, “Grade 2”, “Grade 3”, and “Indeterminate”. In one or more embodiments, an inferencing rule may be applied to determine a fuzzy set membership of a combined output based on the fuzzy set membership of linguistic variables. As a nonlimiting example, membership of a combined output in a “Grade 3” fuzzy set may be determined based on a percentage membership of a second linguistic variable with a first mode in an “Grade 3” fuzzy set and a percentage membership of a second linguistic variable associated with a second mode in a “Grade 2” fuzzy set. In one or more embodiments, parameters or features of medical conditions may then be determined by comparison to a threshold or output using another defuzzification process. Each stage of such a process may be implemented using any type of machine learning model, such as any type of neural network, as described herein. In one or more embodiments, parameters of one or more fuzzy sets may be tuned using machine learning. In one or more embodiments, fuzzy inferencing and/or machine learning may be used to synthesize a plurality of recommended actions. In some cases, outputs such as recommended actions may be combined to make an overall or final determination, e.g., a streamlined version of recommended course of action 172, which may be displayed with or instead of individual outputs. As another nonlimiting example, outputs may be ranked, wherein the output with the highest confidence score may be the output displayed at display device 176 or displayed first in a ranked display of result outputs, consistent with details described above.

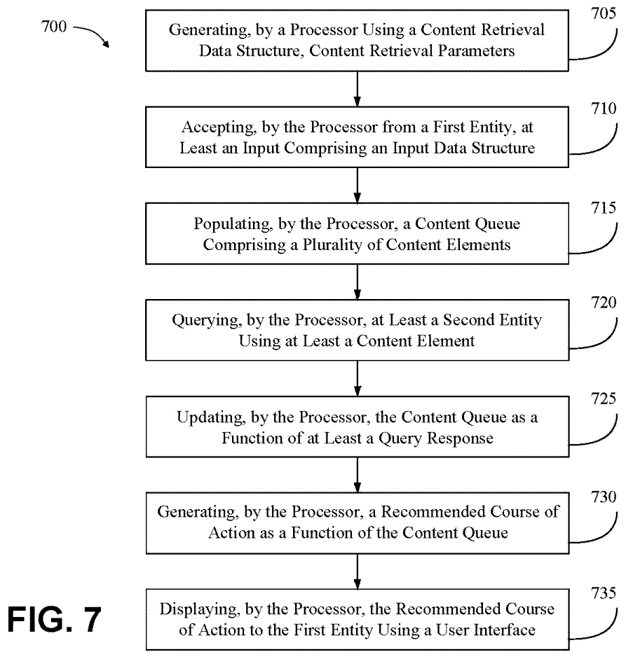

Referring now to FIG. 7, an exemplary embodiment of a method 700 for maintaining retrieved contents is described. At step 705, method 700 includes generating, by processor 104 using at least a content retrieval data structure 116 trained on plurality of training examples 120, plurality of content retrieval parameters 124a-n, wherein generating the plurality of content retrieval parameters 124a-n includes pretraining the at least a content retrieval data structure 116 on a general set of training examples 120 and retraining the at least a content retrieval data structure 116 on a special set of training examples 120, wherein the general and the special set of training examples 120 are subsets of the plurality of training examples 120. This step may be implemented with reference to details described above in this disclosure and without limitation.

With continued reference to FIG. 7, at step 710, method 700 includes receiving, by processor 104 from first entity 128, input data structure 132 as a function of plurality of content retrieval parameters 124a-n. This step may be implemented with reference to details described above in this disclosure and without limitation.

With continued reference to FIG. 7, at step 715, method 700 includes populating, by processor 104 using input data structure 132, content queue 152 comprising plurality of content elements 156a-n. This step may be implemented with reference to details described above in this disclosure and without limitation.

With continued reference to FIG. 7, at step 720, method 700 includes querying, by processor 104, at least a second entity 144 using at least a content element 156a-n of plurality of content elements 156a-n. This step may be implemented with reference to details described above in this disclosure and without limitation.

With continued reference to FIG. 7, at step 725, method 700 includes updating, by processor 104, content queue 152 as a function of at least a query response 160a-n received from at least a second entity 144. This step may be implemented with reference to details described above in this disclosure and without limitation.

With continued reference to FIG. 7, at step 730, method 700 includes generating recommended course of action 172 as a function of content queue 152. This step may be implemented with reference to details described above in this disclosure and without limitation.

With continued reference to FIG. 7, at step 735, method 700 includes displaying recommended course of action 172 to first entity 128 using a user interface. This step may be implemented with reference to details described above in this disclosure and without limitation.

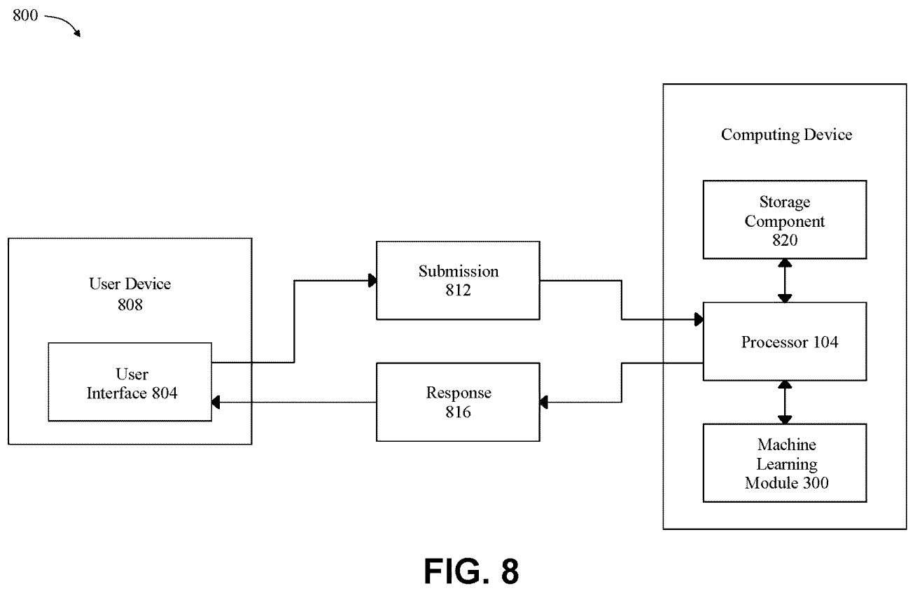

Referring now to FIG. 8, in one or more embodiments, apparatus 100 may perform one or more of its functions, such as accepting input data structure 132 or receiving query response 160a-n, by implementing at least a chatbot system 800, an exemplary embodiment of which is schematically illustrated. In one or more embodiments, a user interface 804 may be communicatively connected with a computing device that is configured to operate a chatbot. In some cases, user interface 804 may be local to computing device. Alternatively, or additionally, in some other cases, user interface 804 may be remote to computing device, e.g., as part of a user device 808, and communicative with the computing device and processor 104 therein, by way of one or more networks, such as without limitation the internet. Alternatively or additionally, user interface 804 may communicate with user interface 804 and/or computing device using telephonic devices and networks, such as without limitation fax machines, short message service (SMS), or multimedia message service (MMS). Commonly, user interface 804 may communicate with computing device using text-based communication, for example without limitation using a character encoding protocol, such as American Standard for Information Interchange (ASCII). Typically, user interface 804 may conversationally interface a chatbot, by way of at least a submission 812, from the user interface 804 to the chatbot, and a response 816, from the chatbot to the user interface 804. In many cases, one or both of submission 812 and response 816 are text-based communication. Alternatively, or additionally, in some cases, one or both of submission 812 and response 816 are audio-based communication.

With continued reference to FIG. 8, submission 812, once received by user interface 804 and/or computing device that operates a chatbot, may be processed by processor 104. In one or more embodiments, processor 104 may process submission 812 using one or more of keyword recognition, pattern matching, and natural language processing. In one or more embodiments, processor 104 may employ real-time learning with evolutionary algorithms. In one or more embodiments, processor 104 may retrieve a pre-prepared response from at least a storage component 820, based upon submission 812. Alternatively, or additionally, in one or more embodiments, processor 104 may communicate a response 816 without first receiving a submission 812, thereby initiating a conversation. In some cases, processor 104 may communicate an inquiry to user interface 804 and/or computing device, wherein processor 104 is configured to process an answer to the inquiry in a following submission 812 from the user interface 804 and/or computing device. In some cases, an answer to an inquiry presented within submission 812 from user device 804 and/or computing device may be used by the computing device as an input to another function.

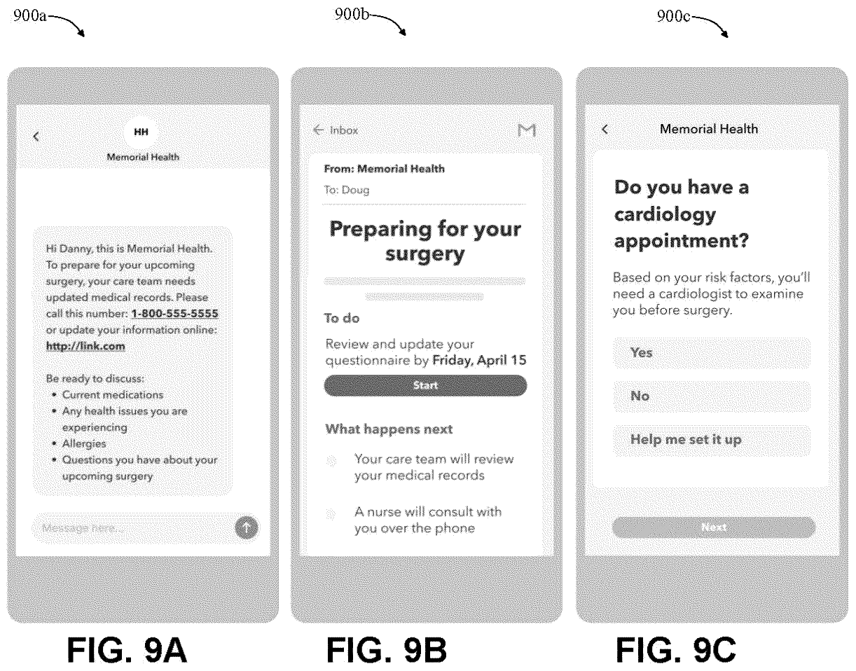

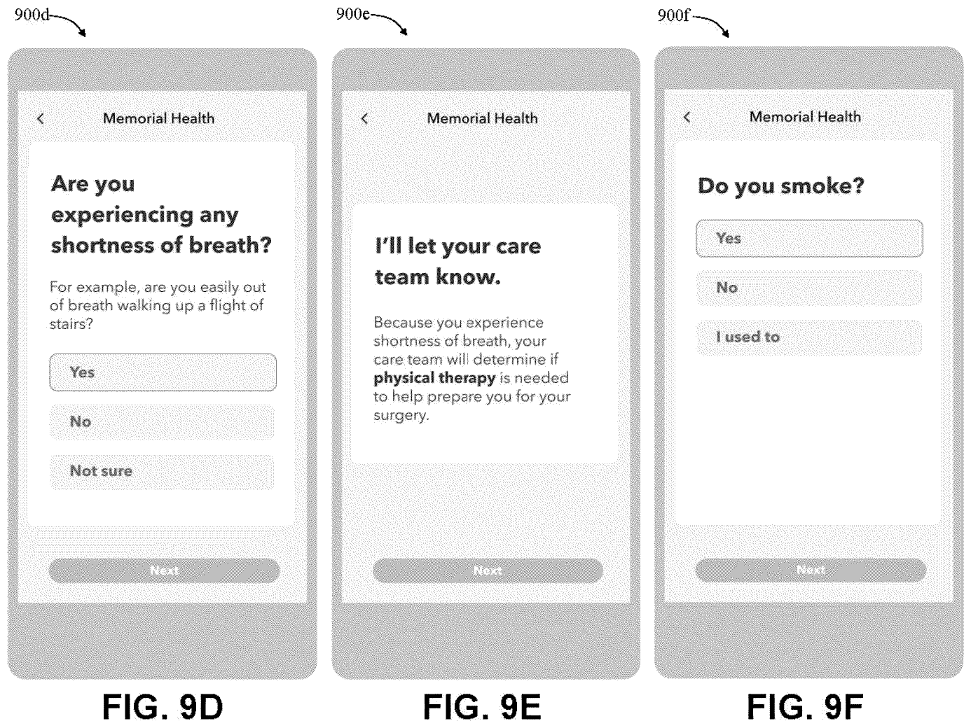

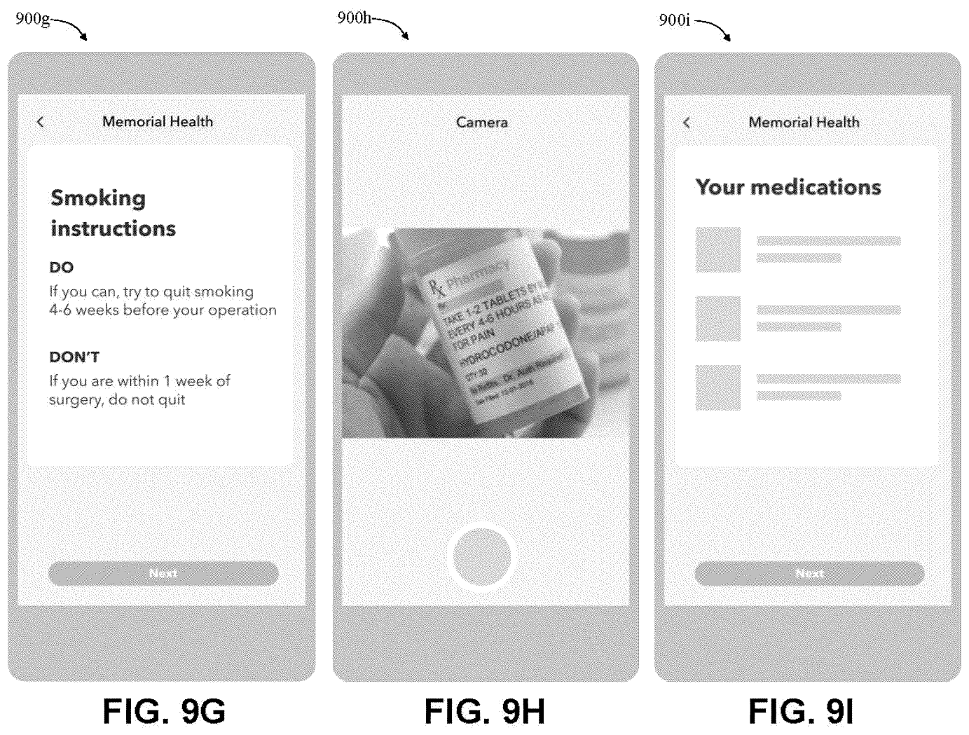

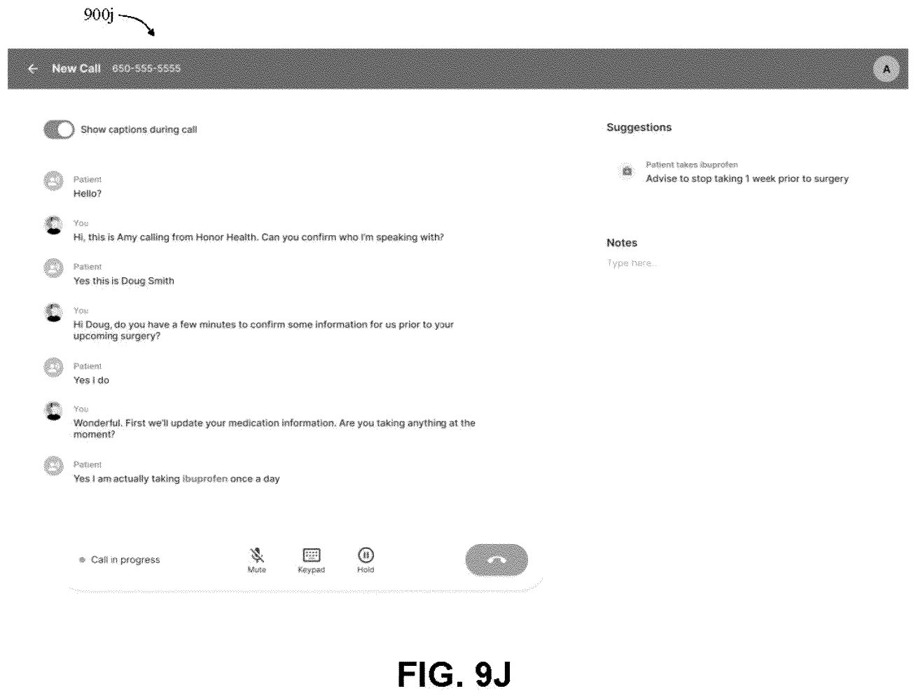

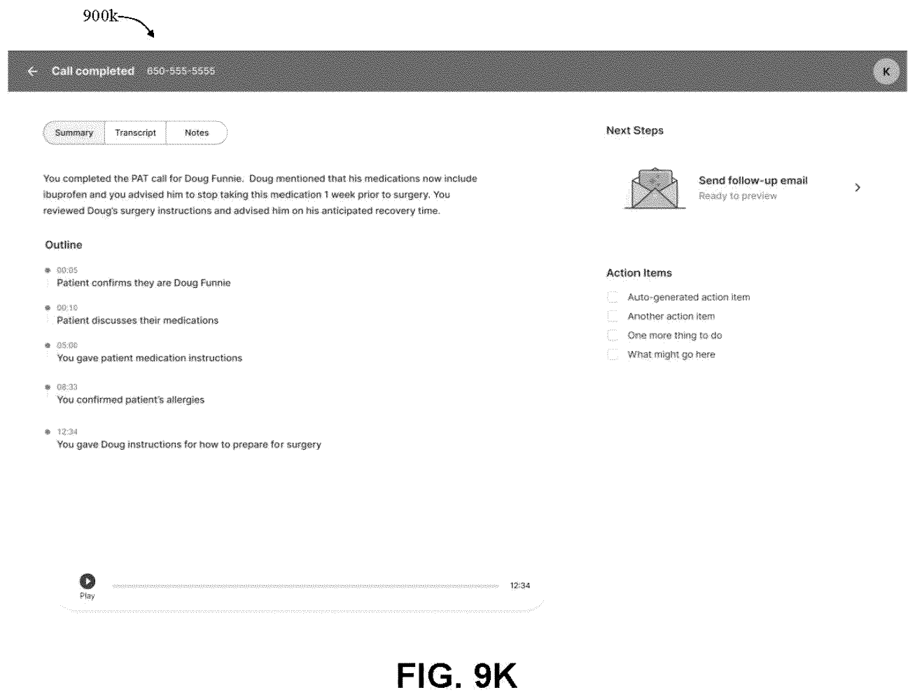

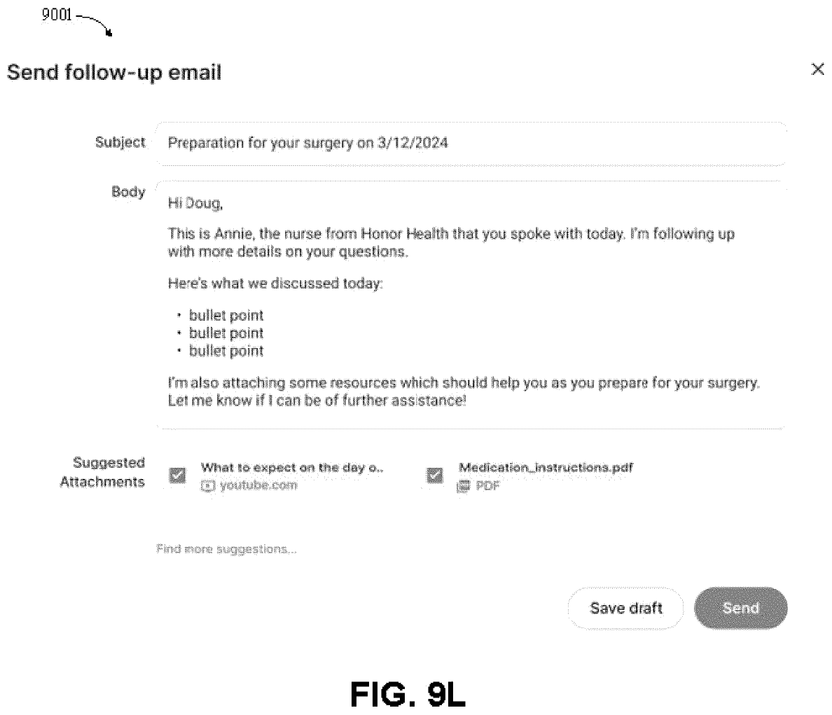

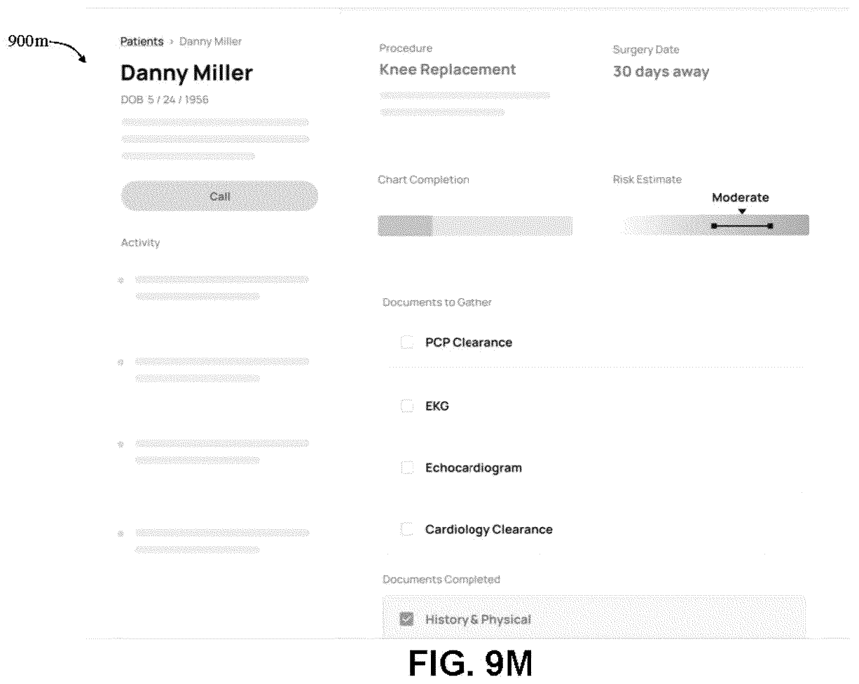

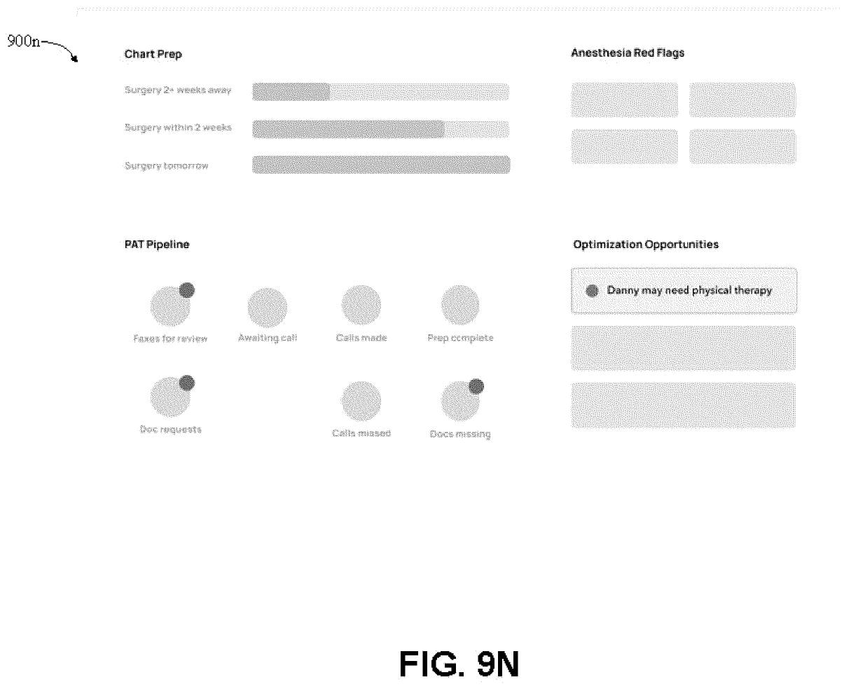

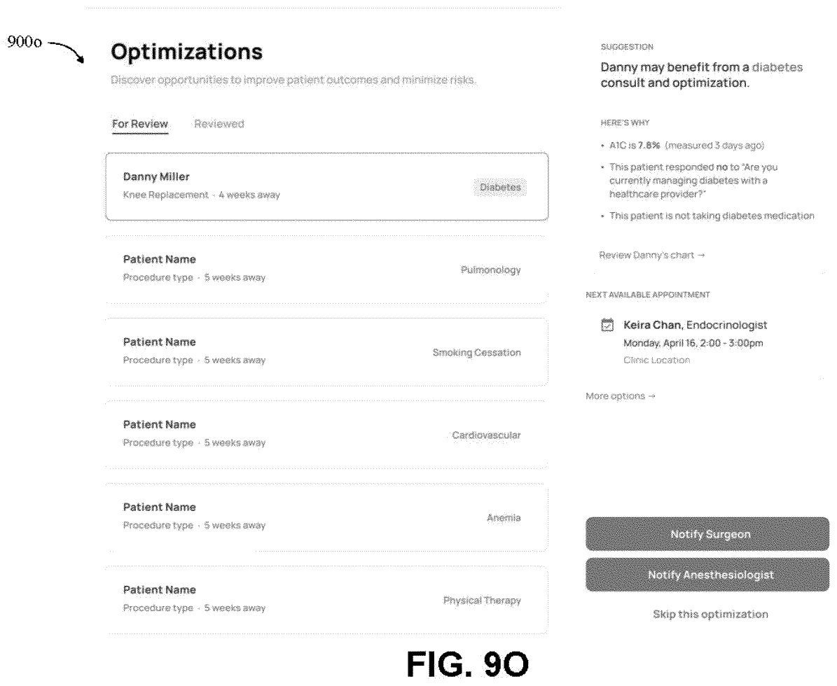

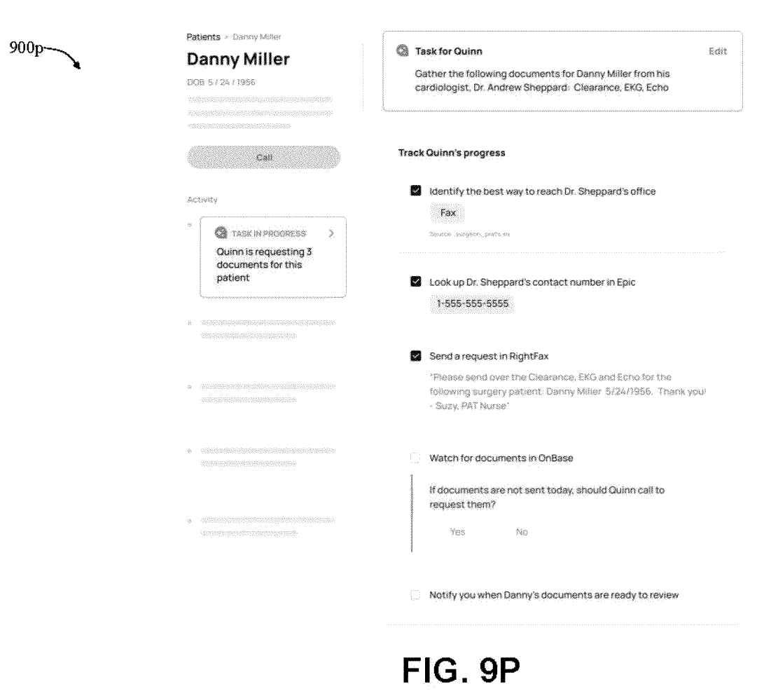

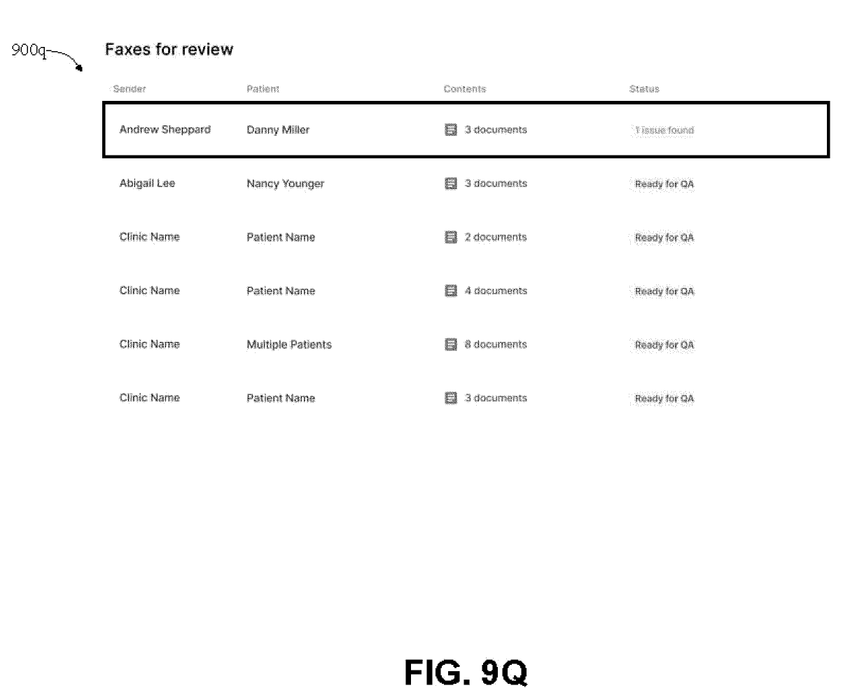

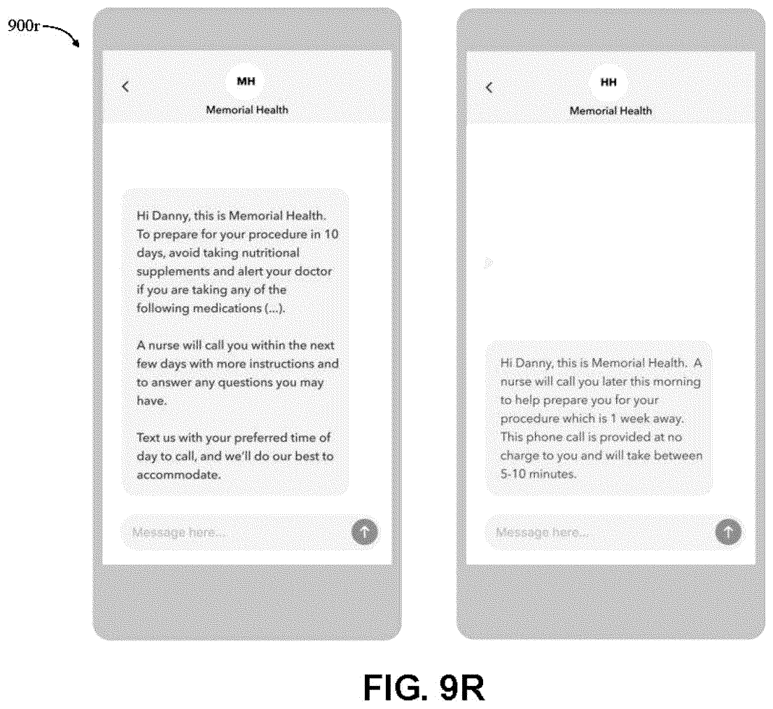

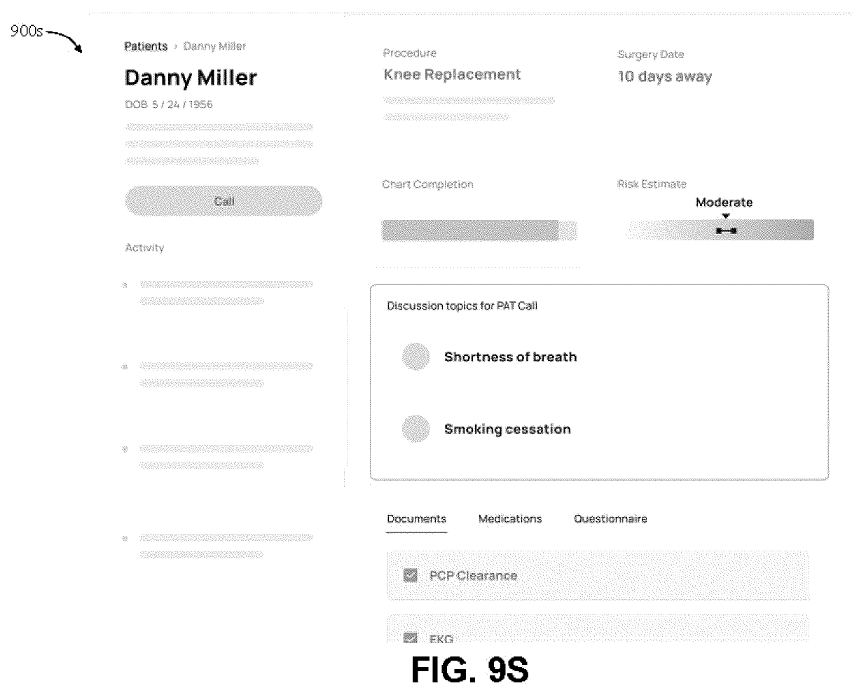

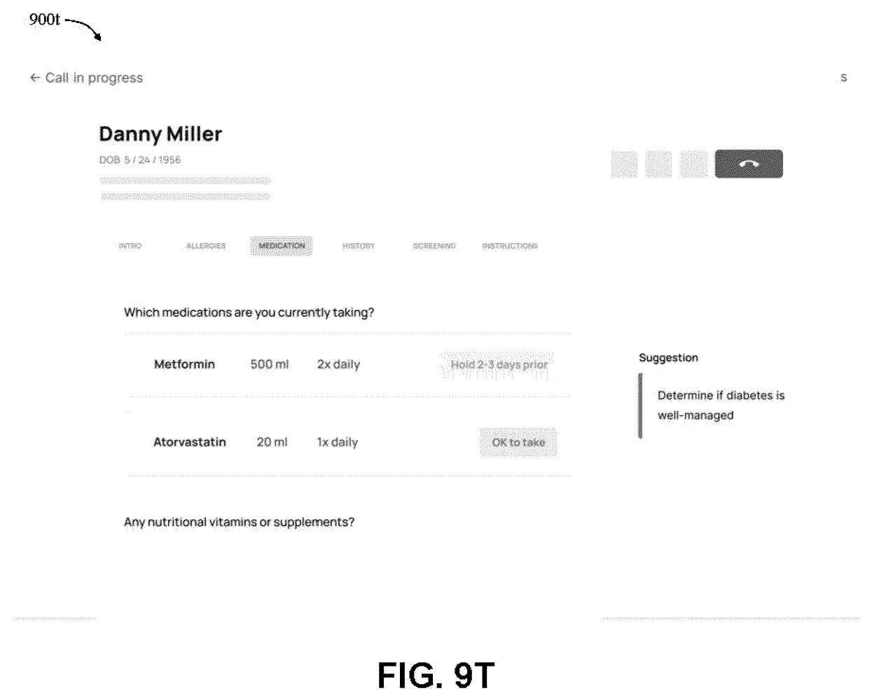

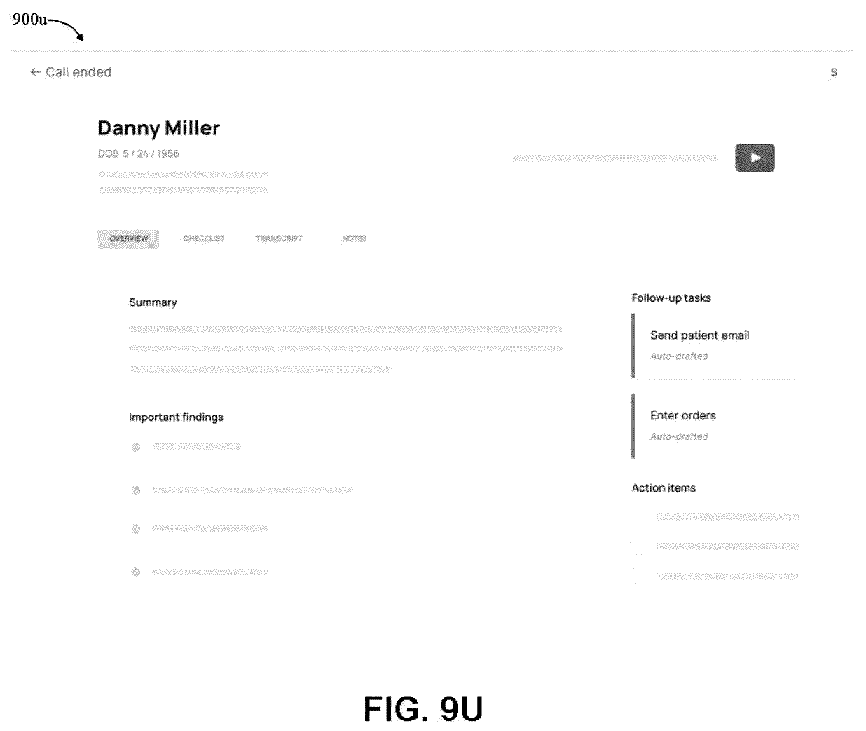

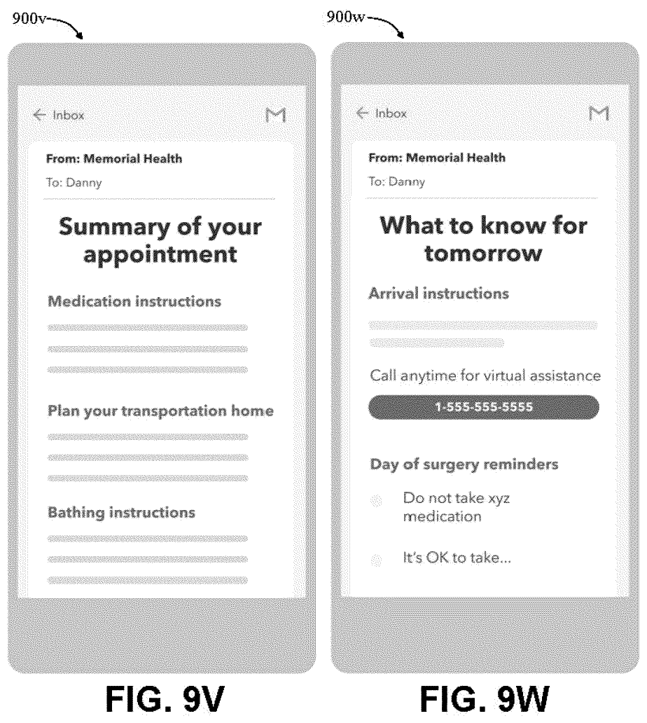

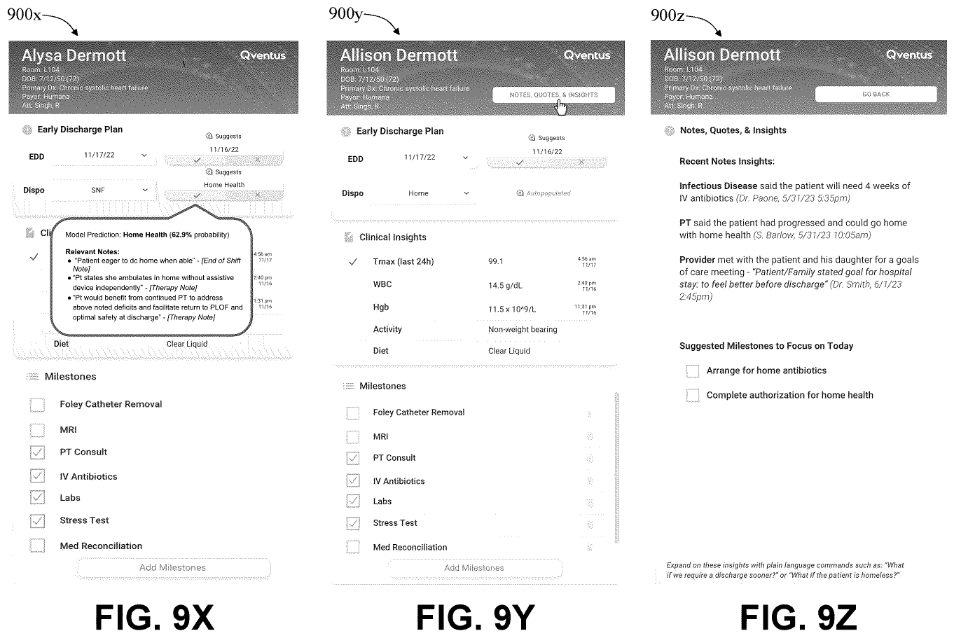

Referring now to FIG. 9A-Z, exemplary GUIs 900a-w are included to collectively illustrate a nonlimiting example of a session orchestrated by apparatus 100.

Referring now to FIG. 9A, GUI 900a includes an exemplary text message sent by apparatus 100 to first entity 128 (in this case, a hypothetical patient named Danny Miller), requesting the first entity 128 to provide one or more input elements of input data structure 132 via phone call and/or online survey/questionnaire. GUI 900a also included a list of items, such as current medications, health issues, allergies, and potential questions regarding an upcoming surgery, that first entity 128 is expected to provide.

Referring now to FIG. 9B, GUI 900b includes an exemplary email sent to first entity 128 (in this case, a hypothetical patient named Doug Smith) requesting one or more input elements of input data structure 132, while providing a list of potential next steps.

Referring now to FIGS. 9C-G, GUIs 900c-g include exemplary items in a survey/questionnaire used by apparatus 100 to request one or more input elements of input data structure 132 from first entity 128. Specifically, GUIs 900c-g include questions regarding cardiology appointment record, shortness of breath symptoms, smoking habits, as well as suggestions and information regarding future steps.

Referring now to FIGS. 9H-I, GUIs 900h-i include exemplary user interfaces wherein first entity 128 may capture and upload a picture of a prescription, using an image capturing device such as a built-in camera of a cell phone, and provide one or more input elements of input data structure 132 by having apparatus 100 extract information from the captured image.

Referring now to FIGS. 9J-L, GUIs 900j-1 include an example of using audio prompt 140 to retrieve secondary input data 216 by placing an automated phone call with first entity 128 (in this case, a hypothetical patient named Doug Smith). GUI 900j includes a transcript of the conversation as well as real-time suggestions and notes. GUI 900k includes a summary of the conversation, organized as a function of a timeline, alongside suggested next steps and action items. GUI 900l includes an exemplary follow-up email for first entity 128 generated by apparatus 100 as a function of secondary input data 216 and/or other information on file, such as suggested next steps and action items.

Referring now to FIGS. 9M-P, GUIs 900m-p includes exemplary content queues 152 generated based on input data structure 132 from one or more first entities 128 (in this case, one of the first entities is a hypothetical patient named Danny Miller). Content queues 152 include information such as a record of activity, scheduled procedure (knee replacement) and date (30 days from now), progress of chart completion, risk estimate (moderate), a list of documents to gather, chart prep progress, anesthesia red flags, pre-admission testing pipeline, optimization opportunities, and lists of tasks to complete.

Referring now to FIG. 9Q, GUI 900q is a list of verification results for a plurality of first entities 128. At least a content disagreement 168 between one or more input elements of input data structure 132 and query response 160a-n is identified for first entity 128 (in this case, a hypothetical patient named Danny Miller).

Referring now to FIG. 9R, GUI 900r includes exemplary text messages sent to first entity 128 (in this case, a hypothetical patient named Danny Miller) that include several reminders as well as information regarding a follow-up phone call from third entity 164 (i.e., a pre-admission testing nurse), in preparation for an upcoming surgery in ten days/one week.

Referring now to FIGS. 9S-T, GUIs 900s-t include exemplary checklists prepared by apparatus 100 that may be used by third entity 164 to conduct a follow-up phone call, as described above. These exemplary checklists highlight key information to review, such as unresolved topics, salient medical history, and communication history with apparatus 100.

Referring now to FIG. 9U, GUI 900u includes a summary of the follow-up phone call with first entity 128 (in this case, a hypothetical patient named Danny Miller) that includes an overview, a checklist, a transcript, notes, as well as summary, important findings, follow-up tasks, and action items.

Referring now to FIG. 9V-W, GUIs 900v-w includes exemplary emails sent to first entity 128 (in this case, a hypothetical patient named Danny Miller) that include personalized summaries and reminders as the date of surgery approaches. GUI 900v includes personalized notes, educational resources, additional reminders, medication instructions, transportation tips, bathing instructions, and/or the like generated using information from GUI 900u. GUI 900w includes arrival instructions, contact information, and day of surgery reminders.

Referring now to FIGS. 9X-Z, GUIs 900x-z include exemplary user interfaces related to first entity 128 (in this case, a hypothetical patient named Alysa Dermott) regarding her milestones of treatment and clinical insights.

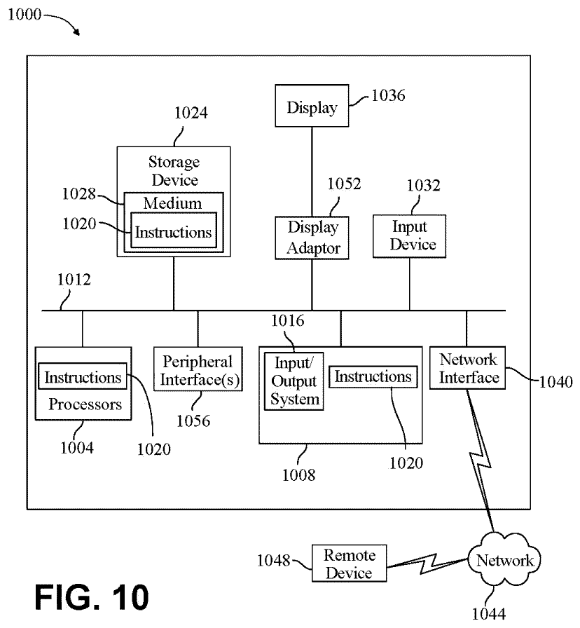

Referring now to FIG. 10, it is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to one of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module. Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random-access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission. Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein. Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

With continued reference to FIG. 10, the figure shows a diagrammatic representation of one embodiment of a computing device in the exemplary form of a computing system 1000 within which a set of instructions for causing the computing system 1000 to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computing system 1000 may include a processor 1004 and a memory 1008 that communicate with each other, and with other components, via a bus 1012. Bus 1012 may include any of several types of bus structures including, but not limited to, a memory bus, a memory controller, a peripheral bus, a local bus, and any combinations thereof, using any of a variety of bus architectures. Processor 1004 may include any suitable processor, such as without limitation a processor incorporating logical circuitry for performing arithmetic and logical operations, such as an arithmetic and logic unit, which may be regulated with a state machine and directed by operational inputs from memory and/or sensors; processor 1004 may be organized according to Von Neumann and/or Harvard architecture as a non-limiting example. Processor 1004 may include, incorporate, and/or be incorporated in, without limitation, a microcontroller, microprocessor, digital signal processor, field programmable gate array, complex programmable logic device, graphical processing unit, general-purpose graphical processing unit, tensor processing unit, analog or mixed signal processor, trusted platform module, a floating-point unit, and/or system on a chip.

With continued reference to FIG. 10, memory 1008 may include various components (e.g., machine-readable media) including, but not limited to, a random-access memory component, a read only component, and any combinations thereof. In one example, a basic input/output system 1016, including basic routines that help to transfer information between elements within computing system 1000, such as during start-up, may be stored in memory 1008. Memory 1008 (e.g., stored on one or more machine-readable media) may also include instructions (e.g., software) 1020 embodying any one or more of the aspects and/or methodologies of the present disclosure. In another example, memory 1008 may further include any number of program modules including, but not limited to, an operating system, one or more application programs, other program modules, program data, and any combinations thereof.

With continued reference to FIG. 10, computing system 1000 may also include a storage device 1024. Examples of a storage device (e.g., storage device 1024) include, but are not limited to, a hard disk drive, a magnetic disk drive, an optical disc drive in combination with an optical medium, a solid-state memory device, and any combinations thereof. Storage device 1024 may be connected to bus 1012 by an appropriate interface (not shown). Example interfaces include, but are not limited to, small computer system interface, advanced technology attachment, serial advanced technology attachment, universal serial bus, IEEE 1394 (FIREWIRE), and any combinations thereof. In one example, storage device 1024 (or one or more components thereof) may be removably interfaced with computing system 1000 (e.g., via an external port connector (not shown)). Particularly, storage device 1024 and an associated machine-readable medium 1028 may provide nonvolatile and/or volatile storage of machine-readable instructions, data structures, program modules, and/or other data for computing system 1000. In one example, software 1020 may reside, completely or partially, within machine-readable medium 1028. In another example, software 1020 may reside, completely or partially, within processor 1004.

With continued reference to FIG. 10, computing system 1000 may also include an input device 1032. In one example, a user of computing system 1000 may enter commands and/or other information into computing system 1000 via input device 1032. Examples of input device 1032 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1032 may be interfaced to bus 1012 via any of a variety of interfaces (not shown) including, but not limited to, a serial interface, a parallel interface, a game port, a USB interface, a FIREWIRE interface, a direct interface to bus 1012, and any combinations thereof. Input device 1032 may include a touch screen interface that may be a part of or separate from display 1036, discussed further below. Input device 1032 may be utilized as a user selection device for selecting one or more graphical representations in a graphical interface as described above.

With continued reference to FIG. 10, user may also input commands and/or other information to computing system 1000 via storage device 1024 (e.g., a removable disk drive, a flash drive, etc.) and/or network interface device 1040. A network interface device, such as network interface device 1040, may be utilized for connecting computing system 1000 to one or more of a variety of networks, such as network 1044, and one or more remote devices 1048 connected thereto. Examples of a network interface device include, but are not limited to, a network interface card (e.g., a mobile network interface card, a LAN card), a modem, and any combination thereof. Examples of a network include, but are not limited to, a wide-area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1044, may employ a wired and/or a wireless mode of communication. In general, any network topology may be used. Information (e.g., data, software 1020, etc.) may be communicated to and/or from computing system 1000 via network interface device 1040.

With continued reference to FIG. 10, computing system 1000 may further include a video display adapter 1052 for communicating a displayable image to a display device, such as display device 1036. Examples of a display device include, but are not limited to, a liquid crystal display (LCD), a cathode ray tube (CRT), a plasma display, a light emitting diode (LED) display, and any combinations thereof. Display adapter 1052 and display device 1036 may be utilized in combination with processor 1004 to provide graphical representations of aspects of the present disclosure. In addition to a display device, computing system 1000 may include one or more other peripheral output devices including, but not limited to, an audio speaker, a printer, and any combinations thereof. Such peripheral output devices may be connected to bus 1012 via a peripheral interface 1056. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. An apparatus for automating pre-procedural coordination workflows in a digital environment, the apparatus comprising:
at least a processor; and
a memory communicatively connected to the at least a processor, wherein the memory comprises instructions configuring the at least a processor to:
sanitize a plurality of training examples comprising medical data, wherein sanitizing the plurality of training examples comprises:
determining that at least one training example entry of the plurality of training examples has a signal to noise ratio below a threshold value; and
removing the at least one training example entry from the plurality of training examples to create a sanitized plurality of training examples;
generate, using at least a content retrieval data structure comprising a machine learning model, a plurality of content retrieval parameters, wherein generating the plurality of content retrieval parameters comprises training the content retrieval data structure on the sanitized plurality of training examples which comprises:
training the at least a content retrieval data structure on a general set of training examples comprising a general collection of medical literature; and
retraining the at least a content retrieval data structure on a special set of training examples comprising a specific medical discipline, wherein the general and the special set of training examples are subsets of the sanitized plurality of training examples;
generate a web query comprising data associated with an input data structure including at least a digital file;
extract textual data from the at least a digital file using a large language model (LLM) wherein the LLM includes an attention mechanism comprising self-attention configured to dynamically quantify features of the at least a digital file by:
searching for a set of positions in a source text where relevant information is concentrated; and
predicting expected text associated with the generated web query based on context vectors associated with the set of positions and previously generated target text comprising textual data of a dictionary correlated to a prompt in a training data set;
receive, from a first entity, the input data structure as a function of the plurality of content retrieval parameters;
populate, using the input data structure, a content queue comprising a plurality of content elements;
query at least a second entity using at least a content element of the plurality of content elements, wherein querying the at least a second entity comprises applying a geofence as a function of a location of the first entity;
update the content queue as a function of at least a query response received from the at least a second entity;
generate a recommended course of action as a function of the updated content queue which comprises:
ranking the plurality of content elements as function of one or more pre-determined criteria comprising temporal sequence; and
generating the recommended course of action based on the rank of the plurality of content elements;
display the recommended course of action which includes the ranking of the plurality of content elements; and
perform the recommended course of action comprising automatically communicating with one or more computing devices to initiate one or more components of the recommended course of action.

2. (canceled)

3. The apparatus of claim 1, wherein:
the input data structure comprises a plurality of input elements; and
receiving the input data structure comprises:
retrieving, from a data repository, a first input element pertaining to the first entity; and
receiving, from the first entity, a second input element as a function of the first input element.

4. The apparatus of claim 3, wherein receiving the input data structure further comprises:
validating the first input element as a function of the second input element; and
updating the input data structure as a function of an outcome of the validation.

5. The apparatus of claim 3, wherein receiving the second input element comprises:
generating, using the at least a content retrieval data structure, at least a prompt as a function of the first input element and the plurality of content retrieval parameters;
receiving, from the first entity, secondary input data in response to the at least a prompt; and
updating the input data structure as a function of the secondary input data.

6. The apparatus of claim 5, wherein receiving the second input element further comprises:
synthetizing, using a speech synthesis algorithm, an audio prompt as a function of the first input element and the plurality of content retrieval parameters;
capturing, using a sound capturing device communicatively connected to the at least a processor, audio secondary input data from the first entity in response to the audio prompt;
transcribing the audio secondary input data into textual secondary input data using the at least a content retrieval data structure; and
updating the input data structure as a function of the textual secondary input data.

7. The apparatus of claim 1, wherein updating the content queue comprises:
confirming, by a third entity, at least a content element of the content queue; and
updating the content queue as a function of an outcome of the confirmation.

8. The apparatus of claim 7, wherein updating the content queue further comprises:
identifying at least a content disagreement by comparing the at least a query response against the at least an input data structure;
annotating the at least a content element as a function of the at least a content disagreement; and
updating the at least a content element by resolving, at the third entity, the at least a content disagreement.

9. (canceled)

10. (canceled)

11. A method for automating pre-procedural coordination workflows in a digital environment, the method comprising:
sanitizing, by at least a processor, a plurality of training examples comprising medical data, wherein sanitizing the plurality of training examples comprises:
determining that at least one training example entry of the plurality of training examples has a signal to noise ratio below a threshold value; and
removing the at least one training example entry from the plurality of training examples to create a sanitized plurality of training examples;
generating, by the at least a processor using at least a content retrieval data structure comprising a machine learning model, a plurality of content retrieval parameters, wherein generating the plurality of content retrieval parameters comprises training the content retrieval data structure on the sanitized plurality of training examples which comprises:
training the at least a content retrieval data structure on a general set of training examples comprising a general collection of medical literature; and
retraining the at least a content retrieval data structure on a special set of training examples comprising a specific medical discipline, wherein the general and the special set of training examples are subsets of the sanitized plurality of training examples;
generating, by the at least a processor, a web query comprising data associated with an input data structure including at least a digital file;
extracting, by the at least a processor using a large language model (LLM), textual data from the at least a digital file using the LLM wherein the LLM includes an attention mechanism comprising self-attention configured to dynamically quantify features of the at least a digital file by:
searching for a set of positions in a source text where relevant information is concentrated; and
predicting expected text associated with the generated web query based on context vectors associated with the set of positions and previously generated target text comprising textual data of a dictionary correlated to a prompt in a training data set;
receiving, by the at least a processor from a first entity, the input data structure as a function of the plurality of content retrieval parameters;
populating, by the at least a processor using the input data structure, a content queue comprising a plurality of content elements;
querying, by the at least a processor, at least a second entity using at least a content element of the plurality of content elements, wherein querying the at least a second entity comprises applying a geofence as a function of a location of the first entity;
updating, by the at least a processor, the content queue as a function of at least a query response received from the at least a second entity;
generating, by the at least a processor, a recommended course of action as a function of the updated content queue which comprises:
ranking the plurality of content elements as function of one or more pre-determined criteria comprising temporal sequence; and
generating the recommended course of action based on the rank of the plurality of content elements;
displaying the recommended course of action which includes the ranking of the plurality of content elements; and
performing, by the at least a processor, the recommended course of action comprising automatically communicating with one or more computing devices to initiate one or more components of the recommended course of action.

12. (canceled)

13. The method of claim 11, wherein:
the input data structure comprises a plurality of input elements; and
receiving the input data structure comprises:
retrieving, from a data repository, a first input element pertaining to the first entity; and
receiving, from the first entity, a second input element as a function of the first input element.

14. The method of claim 13, wherein receiving the input data structure further comprises:
validating the first input element as a function of the second input element; and
updating the input data structure as a function of an outcome of the validation.

15. The method of claim 13, wherein receiving the second input element comprises:
generating, using the at least a content retrieval data structure, at least a prompt as a function of the first input element and the plurality of content retrieval parameters;
receiving, from the first entity, secondary input data in response to the at least a prompt; and
updating the input data structure as a function of the secondary input data.

16. The method of claim 15, wherein receiving the second input element further comprises:
synthetizing, using a speech synthesis algorithm, an audio prompt as a function of the first input element and the plurality of content retrieval parameters;
capturing, using a sound capturing device communicatively connected to the at least a processor, audio secondary input data from the first entity in response to the audio prompt;
transcribing the audio secondary input data into textual secondary input data using the at least a content retrieval data structure; and
updating the input data structure as a function of the textual secondary input data.

17. The method of claim 11, wherein updating the content queue comprises:
confirming, by a third entity, at least a content element of the content queue; and
updating the content queue as a function of an outcome of the confirmation.

18. The method of claim 17, wherein updating the content queue further comprises:
identifying at least a content disagreement by comparing the at least a query response against the at least an input data structure;
annotating the at least a content element as a function of the at least a content disagreement; and
updating the at least a content element by resolving, at the third entity, the at least a content disagreement.

19. (canceled)

20. (canceled)

21. The apparatus of claim 1, wherein the apparatus further comprises
a dedicated hardware unit communicatively connected to the at least a processor,
wherein:
the dedicated hardware unit comprises circuitry configured to perform signal processing operations.

22. The method of claim 11, wherein the method further comprises a dedicated hardware unit communicatively connected to the at least a processor in combination sanitize and further comprise circuitry configured to perform signal processing operations
